(19) United States
(12) Patent Application Publication
(10) Pub. No.: US 2003/0165431 A1
(43) Pub. Date: Sep. 4, 2003

(54) Method for detecting macromolecular conformational change and binding information

(75) Inventors: Alexander Pines, Berkeley, CA (US); David E. Wemmer, Berkeley, CA (US); Megan Spence, Zurich (CH); Seth Rubin, Albany, CA (US); E. Janette Ruiz, Berkeley, CA (US); Ivan E. Dimitrov, North Wales, PA (US)

Correspondence Address: John P. O'Banion, O'BANION & RITCHEY LLP, 400 Capitol Mall, Suite 1550, Sacramento, CA, 95814, US

(73) Assignee: THE REGENTS OF THE UNIVERSITY OF CALIFORNIA

(21) Appl. No.: 10/268,973

(22) Filed: Oct. 9, 2002

Related U.S. Application Data
(63) Continuation-in-part of application No. 09/903,279, filed on Jul. 11, 2001.
(60) Provisional application No. 60/218,549, filed on Jul. 13, 2000.
(60) Provisional application No. 60/399,041, filed on Jul. 25, 2002.
(60) Provisional application No. 60/335,173, filed on Oct. 31, 2001.
(60) Provisional application No. 60/409,410, filed on Sep. 9, 2002.
(60) Provisional application No. 60/335,240, filed on Oct. 31, 2001.

(51) Int. Cl. A61K 49/00; C12Q 1/68

(52) U.S. Cl. 424/9.3; 435/6

(57) ABSTRACT

An active-nucleus sensor or a functionalized active-nucleus complex sensor is utilized in a method for detecting conformational change and binding event information in a targeted molecule, wherein the sensor does not participate in the conformational change or binding event. The method directly or indirectly detects the occurrence, deletion, shift, or any measurable change in a magnetic resonance signal with a unique magnetic resonance property from the active-nuclei, frequently hyperpolarized ¹²⁹Xe.

### CROSS-REFERENCE TO RELATED APPLICATIONS

This application is a continuation-in-part of U.S. application Ser. No. 09/903,279 filed on Jul. 11, 2001 which in turn claims priority from U.S. provisional application serial No. 60/218,549 filed on Jul. 13, 2000.

This application also claims priority from U.S. provisional application serial No. 60/399,041 filed on Jul. 25, 2002, from U.S. provisional application serial No. 60/335,173 filed on Oct. 31, 2001, from U.S. provisional application serial No. 60/409,410 filed on Sep. 9, 2002, and from U.S. provisional application serial No. 60/335,240 filed on Oct. 31, 2001.

This application is related to and incorporates by reference PCT International Publication No. WO 01/05803 A1 published on Jan. 24, 2002.

### STATEMENT REGARDING FEDERALLY SPONSORED RESEARCH OR DEVELOPMENT

This invention was made with Government support under Contract No. DE-AC03-76SF00098 awarded by the Department of Energy, Grant No. RR02305 awarded by National Institutes of Health, and Contract No. N0001498F0402 MDI-II awarded by the Office of Naval Research. The Government has certain rights in this invention.

### REFERENCE TO A COMPUTER PROGRAM APPENDIX

Not Applicable

### BACKGROUND OF THE INVENTION

1. Field of the Invention

An active-nucleus sensor is utilized in a method for detecting conformational change and binding information in a target species, wherein the active-nucleus sensor produces, either directly or indirectly, a detectable signal, NMR or MRI, upon the conformational change and binding in the target species and the sensor does not participate in the conformational change or binding event. More specifically, the subject method employs as the active-nucleus sensor either an active-nucleus gas such as hyperpolarized xenon ¹²⁹Xe, hyperpolarized helium, or sulfur hexafluoride, or active-nuclei ¹⁹F derivatives located in an environment bound to or proximate the target species or a functionalized active-nucleus complex sensor in which an active-nucleus gas such as hyperpolarized xenon ¹²⁹Xe, hyperpolarized helium, or sulfur hexafluoride, or active-nuclei ¹⁹F derivatives are bound in a carrier structure having a binding region specific for the macromolecular target species and upon a conformational change in that target species, often related to a binding event, a detectable signal is produced. The signal that is produced upon the conformational change in the macromolecular target species is any direct or indirect type of created, increased, decreased, shifted, or altered detectable nuclear magnetic resonance signal. Also, the signal may be detectable as a magnetic resonance imaging contrast agent. Further, a plurality of target specific active-nucleus sensors may be utilized in the assaying and screening for conformational changes and binding in samples containing a plurality of targets under either in vivo or in vitro conditions.

2. Description of the Background Art

Xenon has a binding site in the interior of myoglobin (Schoenborn, B. C.; Watson, H. C.; Kendrew, J. C., Nature, 1965, 207, 28-30). Additionally, xenon is relatively small and hydrophobic, and it binds weakly (K≦102 M-1) to the nonpolar interiors of many proteins with little perturbation to the protein structure (Tilton, R. F., Jr.; Kuntz, I. D., Jr.; Petsko, G. A., Biochemistry, 1984, 23, 2849-2857, Prangé, T.; Schiltz, M.; Pernot, L.; Colloc'h, N.; Longhi, S.; Bourget, W.; Fourme, R., Proteins: Struct., Func., Genet., 1998, 30, 61-73, and Quillin, M. L.; Breyer, W. A.; Griswold, I. J.; Matthews, B. W., J. Mol. Biol., 2000, 302, 955-977). The sensitivity of the xenon chemical shift to its local environment (For recent examples see: Labouriau, A.; Panjabi, G.; Enderle, B.; Pietrass, T.; Gates, B. C.; Earl, W. L.; Ott, K. C., J. Am. Chem. Soc., 1999, 121, 7674-7681, Wolber, J.; Cherubini, A.; Dzik-Jurasz, A. S.; Leach, M. O.; Bifone, A., Proc. Natl. Acad. Sci. U.S.A., 1999, 96, 3664-3669, and Brotin, T.; Lesage, A.; Emsley, E.; Collet, A., J. Am. Chem. Soc., 2000, 122, 1171-1174. For a recent review see: Ratcliffe, C. Annu. Rep., NMR Spectrosc., 1998, 36, 124-208.) has motivated magnetic resonance studies of xenon in biological systems (Miller, K. W.; Reo, N. V.; Schoot Uiterkamp, A. J.; Stengle, D. P.; Stengle, T. R.; Williamson, K. L., Proc. Natl. Acad. Sci. U.S.A., 1981, 78, 4946-4949, Tilton, R. F., Jr.; Kuntz, I. D., Jr., Biochemistry, 1982, 21, 6850-6857, and McKim, S.; Hinton, J. F., Biochim. Biophys. Acta, 1994, 1193, 186-198). More recently, the intense ¹²⁹Xe NMR signals attainable with optical pumping techniques (Walker, T. G.; Happer, W., Rev. Mod. Phys., 1997, 69, 629-642) have been used to probe cavities in lyophilized lysozyme and lipoxygenase (Bowers, C. R.; Storhaug, V.; Webster, C. E.; Bharatam, J.; Cottone, A.; Gianna, R.; Betsey, K.; Gaffney, B. J., J. Am. Chem. Soc., 1999, 121, 1, 9370-9377), detect blood oxygenation levels (Wolber, J.; Cherubini, A.; Leach, M. O.; Bifone, A., Magn. Reson. Med., 2000, 43, 491-496), and identify xenon binding sites in a lipid transfer protein (Landon, C.; Berthault, P.; Vovelle, F.; Desvaux, H., Protein Sci., 2001, 10, 762-770) by the spin-polarization induced nuclear Overhauser effect (Navon, G.; Song, Y. Q.; Room, T.; Appelt, S.; Taylor, R. E.; Pines, A., Science, 1996, 271, 1848-1851).

The detection of biological molecules and their interactions is a significant component of modern biomedical research. In current biosensor technologies, simultaneous detection is limited to a small number of analytes by the spectral overlap of their signals. Recent biosensor technologies exploit surface plasmon resonance (M. Malmqvist, Nature 361, 186-187 (1993)), fluorescence polarization (W. J. Checovich, R. E. Bolger, T. Burke, Nature 375, 254-256 (1995)), and fluorescence resonance energy transfer as detection methods (A. Miyawaki et al., Nature 388, 882-887 (1997)). Although the sensitivity of such techniques is excellent, it has proven challenging to extend these assays to multiplexing capabilities because of the difficulty in distinguishing signals from different binding events. While nuclear magnetic resonance (NMR) spectroscopy is able to finely resolve signals from different molecules and environments, the spectral complexity and low sensitivity of NMR spectroscopy normally preclude its use as a detector of molecular targets in complex mixtures. Notable successes (S. B. Shuker, P. J. Hajduk, R. P. Meadows, S. W. Fesik, Science 274, 1531-1534 (1996) and A. Y. Louie et al., Nature Biotechnology 18, 321-325 (2000)) in the application of NMR to such problems are still limited by long acquisition times or a limited number of detectable analytes. Laser polarized xenon NMR benefits from good signal to noise and spectral simplicity with the added advantage of substantial chemical shift sensitivity.

U.S. Pat. No. 5,642,625 discloses a high volume hyperpolarizer for spin-polarized noble gas. A method and apparatus are presented that allow spin exchange between atoms of the noble gas and an alkali metal such as rubidium.

Described in U.S. Pat. No. 5,785,953 is a magnetic resonance imaging technique using hyperpolarized noble gases as contrast agents. In particular, hyperpolarized xenon and helium are utilized in spatial distribution studies.

The foregoing references and patents reflect the state of the art of which the applicant is aware and are tendered with the view toward discharging applicant's acknowledged duty of candor in disclosing information which may be pertinent in the examination of this application. It is respectfully submitted, however, that none of these references/patents teach or render obvious, singly or when considered in combination, applicant's claimed invention.

### BRIEF SUMMARY OF THE INVENTION

An object of the present invention is to disclose a detection method comprising an active-nucleus sensor that generates an NMR and/or MRI detectable signal upon a conformational change in a target molecule or a ligand binding event with the target molecule or a ligand interaction with the targeted molecule, wherein the target and ligand may be of relatively similar molecular size or vastly different molecular size, wherein the sensor does not participate in the conformational change or binding event.

An additional object of the present invention is to describe a conformational change detection method comprising an active nucleus sensor that generates an NMR and/or MRI detectable signal upon a binding event or environmental alteration induced conformational change in a targeted macromolecule, wherein the sensor does not participate in the conformational change or binding event.

Another object of the present invention is to relate a method of in vivo and in vitro detecting/assaying/screening for conformational changes and binding in a targeted macromolecule that comprises either a non-functionalized active-nucleus sensor or a functionalize active-nucleus complex sensor that signals the binding induced conformational change in the targeted macromolecule.

A further object of the present invention is to describe a method of conformational change detection that comprises either a non-functionalized active-nucleus sensor or a functionalized active-nucleus complex sensor that signals the binding induced conformational change in the targeted macromolecule and that may be used in the presence of other non-targeted species.

Still another object of the present invention is to present a conformational change and/or binding detection method in which hyperpolarized ¹²⁹Xe is utilized directly as an active-nucleus sensor or used indirectly in an active-nucleus complex sensor and produces an NMR and/or MRI detectable signal upon a conformational change or a ligand interaction with a targeted biomolecule or a ligand interaction/binding event with the target biomolecule.

Yet a further object of the present invention is to disclose a method for detecting a conformational change and/or binding in a targeted protein in which hyperpolarized ¹²⁹Xe produces an NMR and/or MRI detectable signal upon a conformational change in a targeted protein.

In one embodiment of the subject invention a method is disclosed for detecting conformational change in a macromolecule or a binding event. A useful property of the subject sensor is that the sensor does not participate in the conformational change or binding event, but reports on the conformational change or binding event. The conformational change is generally initiated by either a ligand binding event or an environmental alteration in such factors as: temperature, species concentrations, solvent conditions, ionic strength, and the like. An active-nucleus sensor produces an NMR and/or MRI detectable signal upon a conformational change in a targeted macromolecule or binding event. In particular, ¹²⁹Xe chemical shift depends on the specific conformation of the macromolecule being investigated. The target macromolecules are often biomolecular entities. The target macromolecules may be selected from naturally occurring ones or synthetically produced species and include, but are not limited to: proteins, polynucleotides, polysaccharides, lipids, combinations of the previously listed species, multisubunit complexes or assembles, and the like. By way of illustration and not by way of limitation, maltose binding protein (MBP), ribose binding protein (RBP), glucose/galactose binding protein (GGBP), and nitrogen transcription regulator C (NTRC) are presented to demonstrate conformationally induced ¹²⁹Xe chemical shifts that are detected in the subject method. The ability to discriminate protein conformations through the ¹²⁹Xe chemical shift indicates the utility of ¹²⁹Xe NMR for assessment of protein functional states (conformations) and/or ligand binding events.

Further, a detailed rationale has been found to account for the sensitivity of the non-functionalized sensor, ¹²⁹Xe, to macromolecular conformational changes. The results demonstrate the sensitivity of the ¹²⁹Xe chemical shift to binding affinity and binding site structure. The ability to correlate these properties with their effects on chemical shift lead to a number of applications of ¹²⁹Xe NMR in biochemical and structural studies of proteins. For example, specific xenon-protein interactions can be identified from ¹²⁹Xe chemical shift data alone. This simple assay for xenon binding may be of interest to x-ray crystallographers interested in using xenon to generate heavy atom derivatives; good candidates are proteins with αₙₐₜᵢᵥₑ>α_{denatured} (α is defined in detail later). Because α_{denatured} scales roughly with protein size, it may only be necessary to measure αₙₐₜᵢᵥₑ in order to conclude that a protein binds xenon. Although the use of laser-polarized ¹²⁹Xe facilitates shift measurements by increasing the signal-to-noise ratio, protein titrations can be done with natural ¹²⁹Xe polarization and take only a few hours with standard NMR spectrometers.

The sensitivity of the ¹²⁹Xe chemical shift to MBP conformation and interactions indicates that ¹²⁹Xe NMR can be used as a detector of ligand-binding events and protein conformation in solution. The advantages of ¹²⁹Xe NMR are that reporting is done by a chemically inert, external species in solution, alleviating the need for labeling molecules of interest with radioisotope or fluorescent probes, and biomolecular samples can be recovered in their native state. In addition, ¹²⁹Xe spectra are simple with no background, making them sensitive and easy to analyze. The MBP structure with bound xenon suggests that the maltose induced change in ¹²⁹Xe shift results from the presence of a natural, specific xenon binding site that undergoes a change in structure upon ligand binding. We are currently investigating whether such an effect can be induced by creating xenon binding sites in regions of proteins that have structural differences among conformers. In addition, conformers may be distinguished via the Xe shift due to differences in nonspecific Xe-protein interactions (see, NTRC/glucose binding and FIG. 20).

Also, disclosed is a novel, “functionalized” active-nucleus complex sensor or biosensor that is directed to and signals the presence of a desired biological target species, often of biological origin or importance. An active-nucleus that presents a detectable signal to either nuclear magnetic resonance (NMR) or magnetic resonance imaging (MRI) techniques is utilized in conjunction with a target specific carrier that interacts with both the active-nucleus and a biological target substrate or environment. The active-nucleus is capable of at least a minimal transient binding to a targeting carrier. The targeting carrier associates with the target substrate or environment, thereby stimulating the production of or change in the detectable signal from the active-nucleus in a functionalized interaction. “Functionalized” implies that when the active-nucleus is bound, in at least a minimal transient manner, by the targeting carrier, that the active-nucleus then responds to and signals the association between the targeting carrier and the target substrate or environment.

Since the basic subject invention enables the creation of several extremely powerful and versatile sensors and techniques that have eluded researchers for many years, a number of related embodiments are disclosed below. One requirement for the subject invention is that the reporter nucleus be sufficiently “active” or capable of producing a signal that is detectable by NMR or MRI techniques. Hyperpolarized noble gases such as xenon and helium meet this requirement, as do other nuclei such as ¹⁹F, if present in sufficiently high concentrations. Thus, “active” implies that the nucleus generates a suitable signal that is capable of detection by NMR (either in strong or weak magnetic fields) and/or MRI contrast procedures. Several relatively standard techniques now exist for hyperpolarizing noble gases and include optical pumping or spin exchange procedures.

It is important to appreciate that for the subject invention the signal produced by the functionalized active-nucleus is studied directly to follow the behavior of the biological target substrate or environment. For example, xenon (as indicated above, other suitable active-nuclei are also contemplated as being within the realm of this disclosure), has a chemical shift that is enormously sensitive to its local chemical environment. Tracking of binding and conformational alterations may also be accomplished by following appropriately related NMR signal decreases and relaxation times. With the large xenon NMR signal created by optical pumping, the chemical shift can easily serve as a signature for the different chemical surroundings in which the xenon is found. Direct interaction between xenon and a target molecule has been observed by measuring the chemical shift and relaxation properties on xenon (in particular see, S. M. Rubin, M. M. Spence, B. M. Goodson, D. E. Wemmer, A. Pines, Proceedings of the National Academy of Sciences of the United States of America 97, 9472-9475 (2000) that was part of Provisional Application No. 60/399,041 to which this application claims priority and is specifically incorporated herein by reference). However, the observation of this direct contact may be limited by the weak binding of xenon (or other suitable active-nuclei) to many target molecules of interest. If desired (for example, when no suitable binding of a non-functionalized active-nucleus sensor, such as non-functionalized hyperpolarized ¹²⁹Xe, to the target species exists), to enhance the binding of the sensor, xenon for example, to the biological target species/substrate/molecule/analyte of interest, and thus the population of xenon in contact with the target species/substrate/molecule/analyte, the xenon can be functionalized to strongly bind to the biological target species/substrate/molecule/analyte. This can be achieved by placing the xenon, or other suitable active-nuclei, in a target carrier that chemically recognizes and binds to the target. The target carrier has a first binding region that binds the xenon for at least a minimal transient period (“minimal” in the sense of a sufficiently long period of time to produce a useful signal) or, preferably, very strongly binds the xenon, and can not itself quickly relax the xenon polarization. Amplification of the sensing for both xenon and helium may be achieved by utilizing a “pool” of hyperpolarized active-nuclei atoms that either sense the environment by changes in the functionalized active-nucleus carrier complex (molecule, supramolecular, or microbubble environment) or else are in sufficiently rapid chemical exchange with active-nuclei in biosensor sites that are so sensitive, thereby amplifying the detection intensity yet further.

The target carrier has a second binding region that binds to or reacts with the target species/substrate/molecule/analyte. The target carrier allows xenon, or other active-nuclei, to be held in close proximity to the desired target, giving rise to a signal at a distinctive frequency indicating the presence of the target species/substrate/molecule/analyte. The functionalized active-nucleus/target carrier complex can “recognize” any one of a wide variety of biological target species/substrates/molecules/analytes (virtually an unlimited set of organic/biomolecular structures) including biologically important species such as proteins, nucleic acids, carbohydrates, lipids, metabolites, and the like in either an in vitro or non-invasive in vivo setting at either high or low NMR utilized field strengths. For example, with diseases, the diagnostic power of the subject invention is quite clear. Various diseases present characteristic/defining targets such as unusual membrane proteins, lipids, or carbohydrates, unusual analytes in body fluids, and the like whose presence can easily be detected with the subject invention.

The subject method of assaying and screening for target species in in vivo and in vitro samples/subjects has many strengths, including the large signal to noise ratio afforded by the high polarization achieve with hyperpolarization of xenon, helium, and other suitable nuclei. With xenon, for example, there is a negligible natural presence of xenon, so there would be no interference from background xenon signals. In contrast to fluorescence (and other techniques that generate overlapping or interfering detection signals) assays and screening procedures, multiple functionalized active-nuclei tests are possible in one system (test-tube, plate, microplate, and the like), by creating active-nuclei carriers targeting different species/substrates/molecules/analytes or by altering the structure of the probe itself or both (see below). Each target would give rise to a separate active-nucleus chemical shift. These assays and screenings could also be carried out non-invasively in vivo, avoiding the exposure to radiation that radiometric assays and screenings require. In the case of optical pumping to create hyperpolarization of xenon and helium, because the large active-nuclei signals are generated by the optical pumping, high magnetic fields are unnecessary (as mentioned previously), and the chemical shifts can be detected in low magnetic fields using a superconducting quantum interference device (SQUID).

A preliminary calculation was performed to explore the initial feasibility of the subject technique, which was verified/reduced to practice, as seen in the below cited examples. Based on capabilities of current spectroscopy, 200 nanomoles of nuclear spins are necessary to measure a signal. To compete with or match other forms of assays or screening procedures, 20 picomoles of target species must be detectable, A factor of 10⁴ in signal is required. The hyperpolarization compensates for at least a factor of 10³, and the additional factor of ten is gained by the relatively simplicity of the spectrum, contrasted with a target (protein and the like) spectrum.

Comprising a first embodiment of the subject invention is a method that utilizes an active-nucleus sensor (hyperpolarized ¹²⁹Xe being a useful, though not limiting example) that produces a detectable magnetic signal upon a conformational change or binding event in a targeted macromolecule.

A second embodiment of the subject invention comprises a functionalized active-nucleus sensor that includes an active-nucleus and a target carrier that associates with both the active-nucleus and a desired target species to produce an detectable characteristic signal (typically a chemical shift or relaxation time for NMR or a contrast capability for MRI). The functionalized active-nucleus complex or subject biosensor that may comprise one or more identical or varied second binding regions. Additionally, the functionalized active-nucleus complex or subject biosensor may have varied first binding regions. Also, both the first and second binding regions could be varied within the same subject biosensor. As indicated, the subject invention allows a huge array of possible target species/substrates/molecules/analytes to be assayed/screened for in a parallel or multiplexing detection style within a single sample/subject.

Several possible active-nuclei gases exist, preferable hyperpolarized xenon and hyperpolarized helium, however, ¹⁹F and similar nuclei, in sufficient concentration, are also contemplated. With fluorine atoms, an exemplary functionalized sensor comprises a target carrier having multiple fluorines such as a polyfluorinated dendrimer that selectively binds an organic target species/substrate/molecule/analyte or a form of fluorine such as sulfur hexafluoride trapped/bound within a functionalized (target specific binding) enclosing structure such as in “bubble” or “microbubble” environment as exemplified by a liposome, micelle, vesicle, bucky-ball type structures, natural and synthetic polymeric cages, and like. Conformational changes or alterations in the effective pressure on the “bubble” or “microbubble” would induce detectable signal variations from the subject biosensor. Variations in the immediate vicinity or environment of the biosensor should be detectable and include changes in ion concentrations, functioning of an ion channel, oxygen levels/distribution, neuron activity, and the like. It is noted that hyperpolarized xenon and hyperpolarized helium will also function as the signal reporting active-nuclei within similar functionalized “bubble” or “microbubble” structures.

The first binding region of the targeting carrier interacts/associates/binds with the active-nucleus. This first binding region includes structures such as monoclonal antibodies, other xenon binding proteins, dendrimers, self-assembled lipid complexes, liposomes, cyclodextrins, cryptands, carcerands, microbubbles, micelles, vesicles, molecular tennis balls, fullerenes, many general cage-like structures, and the like.

The second binding region in the targeting carrier comprises that portion of the subject biosensor that interacts with the target species/substrate/molecule/analyte. It is noted that multiple second binding regions are contemplated and may be identical or varied for attachment to a plurality of target sites.

The basic subject biosensor may contain additional useful components/structures. One or more “tether” regions may be included and serve to separate the first and second binding regions and to permit a region that may be further derivatized with additional moieties such as solubilizing regions. The solubilizing regions may contain polypeptides, carbohydrates, and other species that aid in solubilizing the subject probe.

More specifically, a functionalized active-nucleus biosensor is disclosed that capitalizes on the enhanced signal to noise, spectral simplicity, and chemical shift sensitivity of suitable active-nuclei gases (for example only and not by way of limitation, hyperpolarized xenon, hyperpolarized helium, and sulfur hexafluoride) and polyfluorinated containing species (utilized to target organic molecules) to detect specific targets. One subject sensor embodiment utilizes laser polarized xenon “functionalized” by a biotin-modified supramolecular cage, including a tether region having a solubilizing region, to detect biotin-avidin binding. This biosensor methodology can be used in analyte assays and screening procedures or extended to multiplexing assays for multiple analytes of screenings for multiple species.

Other objects, advantages, and novel features of the present invention will become apparent from the detailed description that follows, when considered in conjunction with the associated drawings.

### BRIEF DESCRIPTION OF THE DRAWINGS

The invention will be more fully understood by reference to the following drawings which are for illustrative purposes only:

FIG. 1 is a schematic model showing a first embodiment of the subject sensor illustrating a first binding region for holding the active-nucleus and a second binding region that associates with the target.

FIG. 2 is the schematic model sensor shown in FIG. 1 with a target species bound to the second binding region.

FIG. 3 is a schematic model showing a second embodiment of the subject sensor illustrating a first binding region identical to that depicted in FIG. 1 and a varied second binding region.

FIG. 4 is a schematic model showing a third embodiment of the subject sensor illustrating a varied first binding region and a second binding region identical to that depicted in FIG. 1.

FIG. 5 is a schematic model showing a fourth embodiment of the subject sensor illustrating both a varied first binding region and a varied second binding region, relative to those seen in FIG. 1.

FIG. 6 is a schematic model showing a fifth embodiment of the subject sensor illustrating a first binding region and a plurality of varied second binding regions.

FIG. 7 is a schematic model showing a sixth embodiment of the subject sensor illustrating a polyfluorinated first binding region and a second binding region that associates with an organic target species.

FIG. 8 is a schematic model showing a seventh embodiment of the subject sensor illustrating a first binding region containing sulfur hexafluoride and a second binding region.

FIG. 9A is a schematic model showing an eighth embodiment of the subject sensor illustrating a first binding region for holding the active-nucleus, a second binding region that associates with the target, and a tether region that connects the first and second binding regions.

FIG. 9B is the eighth sensor embodiment, seen in FIG. 9A, bound to a target species.

FIG. 10 is the eighth sensor embodiment, seen in FIG. 9A, illustrating an active-nuclei exchange process that enhances the generated detection signal.

FIG. 11 is a specific chemical structure of the subject sensor, without an active-nucleus, showing a first binding region (cryptophane-A) for holding the active-nucleus, a second binding region that associates with the target, a tether region that connects the first and second binding regions, and a solubilizing polypeptide attached to the tether.

FIG. 12 is the specific chemical structure shown in FIG. 11 with the active-nucleus xenon included within the cage-like, cryptophane-A, first binding region.

FIG. 13 shows ¹²⁹Xenon NMR spectra that monitors the binding of a biotin-functionalized xenon biosensor to avidin.

FIG. 14 shows the effect of cage structure (cryptophane-A in the top “A” view and cryptophane-E in the bottom “B” view) on the bound xenon chemical shift.

FIG. 15 is a schematic diagram showing multiplexing with functionalized xenon biosensors in which the top spectrum shows the three distinct functionalized xenon peaks, corresponding to different first binding region cages tethered to three second binding region ligands. The bottom spectrum shows the effect of adding the functionalized xenon biosensor to an unknown sample solution having targets.

FIG. 16 is a ¹²⁹Xe spectra of laser-polarized xenon dissolved in solution containing 350 μM maltose binding protein (MBP) in the absence of ligand (a) and in the presence of 1 mM β-cyclodextrin (b), and 1 mM maltose (c).

FIG. 17 shows ¹²⁹Xe chemical shift data as a function of maltose binding protein (MBP) concentration in solution containing buffer alone (▪) and buffer with 1 mM β-cyclodextrin (♦), and 1 mM maltose (●).

FIG. 18 shows ¹²⁹Xe NMR spectra of laser-polarized xenon dissolved in the crude cell lysates from two samples of E. coli with (a) and without (b) the expression plasmid for MBP.

FIG. 19 shows ¹²⁹Xe chemical shift data for both ribose binding protein (RBP) as a function of RBP concentration in solution containing buffer alone (♦) and buffer with 1 mM ribose (▴) and glucose/galactose binding protein (GGBP) as a function of GGBP concentration in solution containing buffer alone (●) and buffer with 1 mM glucose (▪).

FIG. 20 shows ¹²⁹Xe chemical shift data for nitrogen transcription regulator (NTRC) as a function of NTRC concentration for the activated (NTRC BeFₓ) form (▪) and the inactivated form (♦).

FIG. 21 shows the effects of the maltose binding protein (MBP) on ¹²⁹Xe chemical shifts. (a) ¹²⁹Xe NMR spectra of 1 mM xenon in solution with MBP at varying concentrations. The changes in ¹²⁹Xe chemical shift and resonance line width results from increased xenon-protein interactions with concentration. (b) ¹²⁹Xe chemical shift titration data for 1 mM (●) xenon and 7 mM xenon (◯) in solution with MBP under native conditions and 10 mM in solution with MBP under denaturing conditions (▪). The slope of both titrations under native conditions are similar (α=2.5±0.1 ppm/mM for 1 mM xenon and α=2.4±0.1 ppm/mM for 4 mM xenon), indicating that a small fraction of xenon interacts with any one protein site. The fact that the slope under denaturing conditions (α=1.3±0.1 ppm/mM) is less than under native conditions suggests the presence of a specific xenon binding site. The reported ¹²⁹Xe chemical shifts for each titration are referenced to the shift of xenon in buffer.

FIG. 22 shows a stereo diagram of MBP with bound xenon. The density shown in magenta marks the difference Fourier map (contoured at 5σ) generated from the previously published native data set and the data set presented here for MBP crystals pressurized with 8 atm xenon. The xenon binding cavity is located in the N-terminal domain of the protein below the surface of the sugar binding cleft.

FIG. 23 shows a stereo view of the xenon binding cavity in the maltose binding protein. The electron density (lattice pattern) corresponds to a 2Fₒ-F_{c}map contoured at 2σ. The xenon is localized in the cavity away from the lysine and water that mark the end of the cavity at the protein surface. While the presence of many hydrophobic side chains is often observed in xenon binding cavities, the proximity of the cavity to the surface is less common.

FIG. 24 shows Evidence for a difference in xenon binding affinity between the β-cyclodextrin and maltose complexes of MBP. (a-b) Portions of the ¹H-¹⁵N HSQC correlation spectra of 0.4 mM MBP in solution with 2 mM β-cyclodextrin and no xenon (each tagged with a thick tail/line) and 72 mM xenon (each tagged with a thin tail/line). The resonances that have previously been assigned to Leu262 and Gly16 shift with increasing xenon concentration; in the crystal structure, these resonances line the cavity where xenon is bound. (c-d) Portions of the ¹H-¹⁵N HSQC correlation spectra of 0.4 mM MBP in solution with 2 mM maltose and no xenon (each tagged with a thick tail/line) and 72 mM xenon (each tagged with a thin tail/line). The lack of significant shift changes for the same resonances indicates a weaker affinity of xenon for the maltose complex at the binding site observed in the crystal structure. (e) Plots of the total change in chemical shift as a function of xenon concentration for the resonances shown in a (◯) and b (●). Fits of the data to a two-site model yield an association constant of 20±10 M⁻¹ for xenon bound to the β-cyclodextrin complex.

FIG. 25 shows the effect of changing xenon binding site structure on ¹²⁹Xe chemical shifts. (a) ¹²⁹Xe NMR spectra of 1.5 mM xenon in solution with 0.3 mM WT* and L121 T4 lysozyme. (b) Plot of the changes in ¹²⁹Xe chemical shift as a function of protein concentration for xenon in solution with WT* (▪) and L121A (●). The difference in slope between the WT* titration (α=2.3±0.1 ppm/mM) and the L121A titration (α=0.9±0.1 ppm/mM) results from different contributions to the chemical shift from the xenon binding site in each protein. The smaller WT* cavity induces a larger downfield shift than the larger L1212A cavity.

FIG. 26 shows the effects of inhibiting xenon binding sites on ¹²⁹Xe chemical shifts. (a) ¹²⁹Xe NMR spectra of 1.5 mM xenon in solution with 0.3 mM L99A T4 lysozyme and L99A with benzene and n-butylbenzene. (b) Plot of the changes in ¹²⁹Xe chemical shift as a function of protein concentration for xenon in solution with L99A alone (▪) and L99A with benzene (●) and n-butylbenzene (▴). The difference in slope between the L99A titrations without (α=2.7±0.1 ppm/mM) and with benzene (α=2.4±0.1 ppm/mM) corresponds to the contribution from xenon-protein interactions at site 1, while the difference in slope between the benzene and n-butylbenzene (α=0.9±0.1 ppm/mM) titrations corresponds to the contribution from interactions at site 2.

FIG. 27 shows a determination of xenon-L99A binding affinities. (a and b) Portions of the ¹H-¹⁵N HSQC correlation spectra of 1.2 mM L99A T4 lysozyme in solution alone (no tag), with benzene (each tagged with a thick tail/line), with 77 mM xenon (each tagged with a medium tail/line), and with both benzene and 105 mM xenon (each tagged with a thin tail/line). Resonances that show changes in chemical shift with addition of xenon correspond to protons near the xenon binding sites. Peaks with significant chemical shift changes upon addition of benzene (a) correspond to amide protons near site 1, while resonances with small shifts (b) correspond to protons near site 2. (c) Plots of the total change in chemical shift as a function of xenon concentration for the resonances shown in (a) and (b). Fits of the data to a two-site model yield an association constant of 90±10 M⁻¹ for xenon bound to site 1 in the absence of benzene (●) and 30±10 M⁻¹ for xenon bound to site 2 in the presence of benzene (▪).

FIG. 28 shows a comparison of the structure of the xenon binding cavity in MBP in the β-cyclodextrin (tagged with a line-square) and maltose (tagged with a line-circle) complexes. The cavity space in the β-cyclodextrin structure is shown in magenta. The position of most of the residues that line the cavity is similar except for Lys15, which moves to hydrogen bond to the reducing glycosyl unit of maltose. This subtle change in the cavity likely accounts for the sensitivity of the ¹²⁹Xe chemical shift to the conformation of MBP.

### DESCRIPTION OF THE PREFERRED EMBODIMENT(S)OF THE INVENTION

¹²⁹Xenon has magnetic resonance properties, such that the chemical shift and spin-lattice relaxation rate that are enormously sensitive to its local environment. In aqueous solution, the xenon chemical shift has been shown to be affected by preferential binding interactions and weak, diffusion mediated interactions between xenon and other solutes, including macromolecules such as proteins. In the case of interactions with proteins, for example, the chemical shift of xenon will change with a change in any of the following properties of the protein and xenon-protein interaction: protein concentration, quantity and chemical identity of protein accessible surface area, amino acid composition, number of specific xenon binding sites in protein, size and shape of xenon binding sites, and strength of interaction between xenon and protein at binding sites. Accordingly, for the subject invention and described below in detail, a change in a protein's three-dimensional structure (macromolecular conformational change with external and internal dimensional alterations usually occurring) has been observed to result in a change in the xenon chemical shift.

The use of xenon as a sensor of macromolecular conformational changes has a variety of applications. Binding interactions between proteins and small molecule ligands, other proteins, and other macromolecules such as nucleic acids and polynucleic acids often result in a conformational change; therefore, xenon can be used as a sensor of the presence of such interactions or a change in these interactions upon changing solution conditions. The subject sensor does not participate in the conformational change or binding event and thus reports without interfering or influencing in the observed process or processes. The detection of small molecule ligand binding is an important assay used in a number of drug discovery methods; the use of xenon chemical shifts to detect drug binding has features that meet the requirements of modern high-throughput drug screening. Conformational changes of proteins that occur upon ligand binding should be detectable in the range of 10-100 micromolar concentrations of ligand and protein. The use of optical pumping and microanalytical NMR techniques can allow for the detection of xenon chemical shifts in sample volumes less than a microliter without the need for time-consuming signal averaging. A large number of samples can be assayed through the use of an array of NMR detection coils or special localization of samples in a single coil using MRI techniques.

The use of xenon chemical shifts as a method of screening for ligand binding has additional features that are advantageous over current drug screening techniques. Detection of free xenon in solution alleviates the need to chemically modify either ligands or proteins with fluorescence or radioisotope probes. Furthermore, the fact that modification of the protein target is unnecessary implies that no prior knowledge of the locations of ligand binding is needed to detect the binding interaction. The fact that ligand binding to a protein target results in a characteristic change in xenon chemical shift that is particular to each protein suggests that multiple protein targets can be assayed. Additionally, targets can be assayed in the presence of other proteins or molecules in solution, which alleviates the need for purification of the target; accordingly, assays can be conducted on cell lysates or with in vitro expression products. Finally, each ligand has a effect on the xenon chemical shift that can be separated from the effects of protein targets and other ligands, allowing for the use of many different ligands in the same assay.

A characteristic change in the xenon chemical shift upon the interaction of a protein with a ligand can also be used as a means of detecting the presence of either analyte in the presence of other molecules in solution. This methodology can be useful in assays, either in vitro or in vivo, that detect protein or metabolic concentration levels. Through the use of MRI techniques, protein and metabolite levels can be assayed and localized spatially within a cell, organism, and the like.

Additionally, Xenon binding sites in proteins have lead to a number of applications of xenon in biochemical and structural studies. Here we further develop the utility of ¹²⁹Xe NMR in characterizing specific xenon-protein interactions. The sensitivity of the ¹²⁹Xe chemical shift to its local environment and the intense signals attainable by optical pumping make xenon a useful NMR reporter of its own interactions with proteins. A method for detecting specific xenon binding interactions by analysis of ¹²⁹Xe chemical shift data is illustrated using the maltose binding protein (MBP) from Escherichia Coli as an example. The crystal structure of MBP in the presence of 8 atm of xenon confirms the binding site determined from NMR data. Changes in the structure of the xenon binding cavity upon the binding of maltose by the protein can account for the sensitivity of the ¹²⁹Xe chemical shift to MBP conformation. ¹²⁹Xe NMR data for xenon in solution with a number of cavity containing T4 lysozyme mutants show that xenon can report on cavity structure. In particular, a correlation exists between cavity size and the binding induced ¹²⁹Xe chemical shift. Further applications of ¹²⁹Xe NMR to biochemical assays, including the screening of proteins for xenon binding for crystallography, are considered.

Functionalized Active Nucleus Sensor

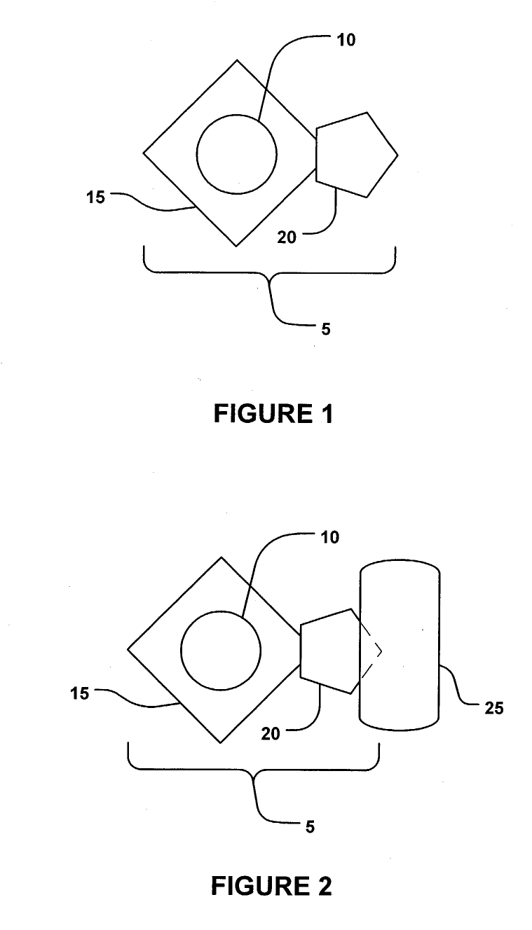

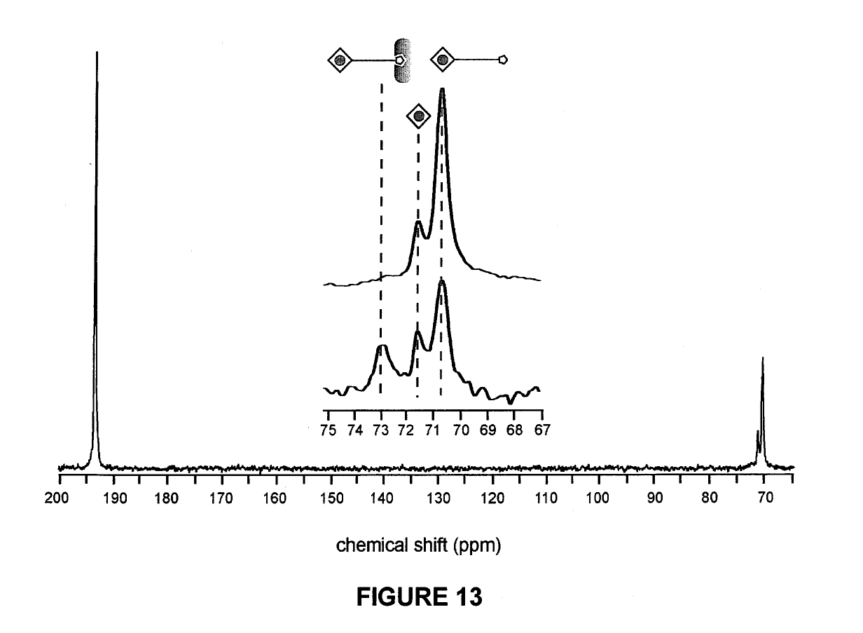

One preferred embodiment of the subject invention is disclosed in the specification and depicted in FIGS. 1-15. This embodiment's most basic configuration comprises an active-nucleus (NMR or MRI detectable nuclei, preferably hyperpolarized xenon or hyperpolarized helium, however, ¹⁹F is useful if present at sufficient levels) and a target carrier that associates with both the active-nucleus and a desired target to produce an detectable characteristic signal (typically a chemical shift or relaxation time for NMR or a contrast capability for MRI). FIG. 1 depicts the basic biosensor (the functionalized active-nucleus complex) 5 configuration and FIG. 2 shows the basic functionalized biosensor 5 bound to a target species/substrate/molecule/analyte 25. An active-nucleus 10 is bound in a targeting carrier. The targeting carrier comprises, at least, a first binding region 15, binding the active-nucleus, and a second binding region 20, wherein the second binding region 20 has a binding affinity for the target species/substrate/molecule/analyte 25 (the dashed line indicating the binding domain in the target). The detectable signal generated from the bound complex 5 in FIG. 2 is distinguishable from the detectable signal produced from the unbound complex 5 in FIG. 1 (see FIG. 13 for an equivalent signal shift).

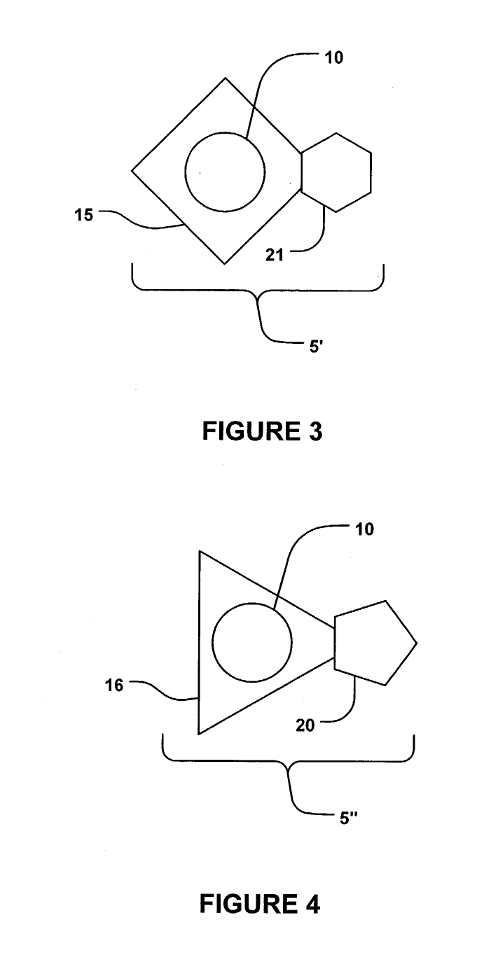

As indicated above, one extremely useful characteristic of the subject invention is that the signal produced from the subject sensor is highly dependent upon its immediate environment and that signals created from similar, but not identical, sensors can be distinguished and utilized to detect multiple target species/substrates/molecules/analytes within the same sample. For example, FIG. 3 depicts a functionalized active-nucleus complex or subject biosensor 5′ that has a varied second binding region 21, relative to the second binding region 20 seen in FIGS. 1 and 2. Thus, biosensor 5′ would bind to a different target species/substrate/molecule/analyte or a different location on the original target species/substrate/molecule/analyte 25.

Additionally, FIG. 4 illustrates a functionalized active-nucleus complex or subject biosensor 5″ that has a varied first binding region 16, relative to the first binding region 15 seen in FIGS. 1 and 2. Thus, biosensor 5″ would generate a different signal than the signal produced by biosensor 5.

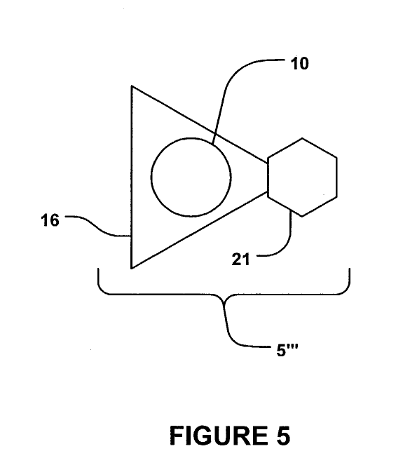

Also, both the first 16 and second binding regions 21 could be varied, relative to the biosensor seen in FIGS. 1 and 2, within the same subject biosensor to form biosensor 5′″, as seen in FIG. 5. As indicated, the subject invention allows a huge array of possible target species/substrates/molecules/analytes to be assayed/screened for in a parallel or multiplexing detection style within a single sample.

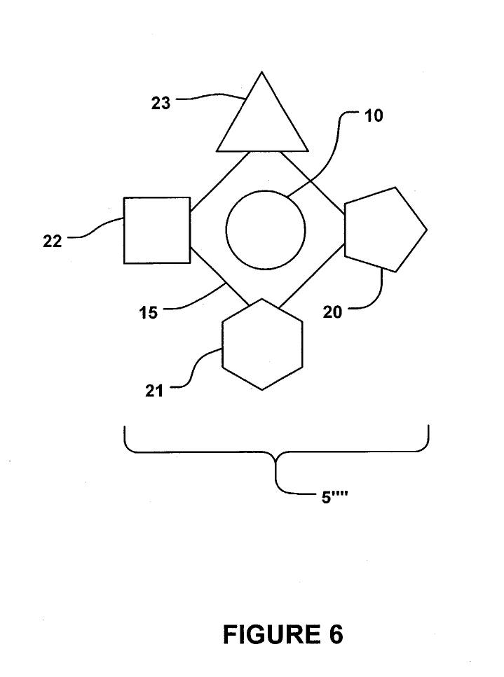

FIG. 6 illustrates a subject biosensor that has several different second binding regions 20, 21, 22, and 23 attached to a first binding region producing sensor 5″″. Sensor 5″″ may bind to one or more targets via the presented second binding regions.

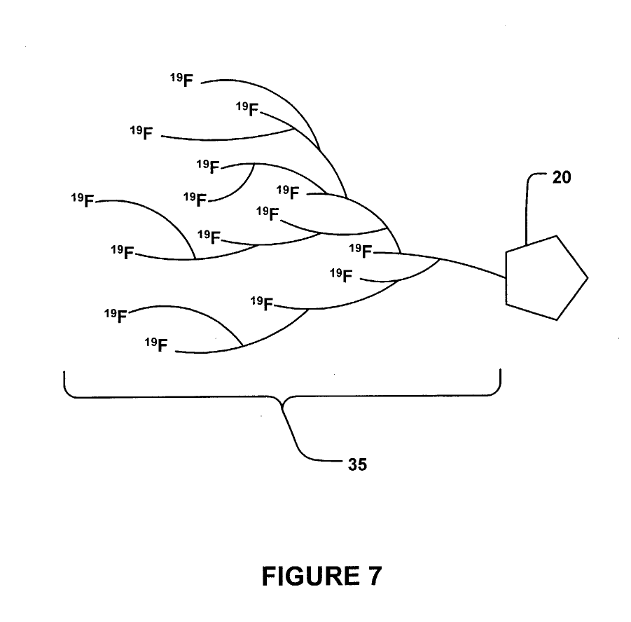

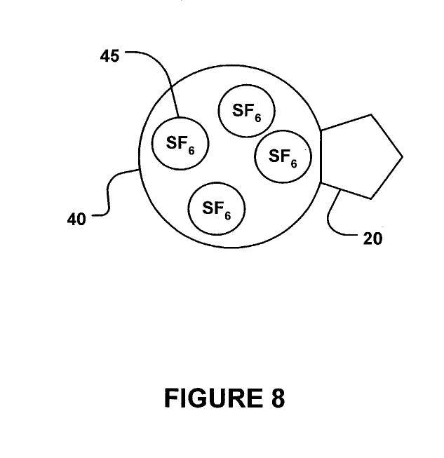

As indicated, several possible active-nuclei gases exist for any target species, preferable hyperpolarized xenon, hyperpolarized helium, and sulfur hexafluoride, however, ¹⁹F, in sufficient concentration, is also contemplated for organic/biological targets. With fluorine atoms, an exemplary functionalized sensor comprises a target carrier having multiple fluorines such as a polyfluorinated dendrimer that selectively binds an organic/biological target species/substrate/molecule/analyte, as seen in FIG. 7. The polyfluorinated first binding region 35 may be a dendrimer or other suitable structure, including, but not limited to natural and synthetic polymers and the like. Additionally, sufficient fluorine to produce an acceptable signal may be in the form of fluorine in sulfur hexafluoride and similar compounds. FIG. 8 illustrates sulfur hexafluoride 45 trapped/bound within a functionalized (target specific binding) enclosing structure 40 such as in “bubble” or “microbubble” environment as exemplified by a liposome, micelle, vesicle, bucky-ball type structures, natural and synthetic polymeric cages, and the like. A second binding region 20 is coupled to the enclosing structure 40 and binds the target. Conformational changes or alterations in the effective pressure on the “bubble” or “microbubble” would induce detectable signal variations from the active-nucleus in a subject biosensor. Variations in the immediate vicinity of the biosensor should be detectable and include such changes as: ion concentrations, oxygen levels, neuron activity, and the like. It is noted that hyperpolarized xenon and hyperpolarized helium will also function as signal reporting active-nuclei within similar functionalized “bubble” or “microbubble” structures.

It is noted that the subject targeting carrier comprises the first binding region (15 and 16 in FIGS. 1-6) that interacts/associates/binds with the active-nucleus. This first binding region includes structures such as monoclonal antibodies, other xenon binding proteins, dendrimers, self-assembled lipid complexes, liposomes, cyclodextrins, cryptands, cryptophanes, carcerands, microbubbles, micelles, vesicles, molecular tennis balls, fullerenes, many general cage-like structures, and the like. As long as structure or chemical nature of the first binding region permits effective signal producing interactions with the active-nucleus and binding to the target is not negated, a wide range of acceptable structures exists for this portion of the subject biosensor (the chemical shifts or relaxation times for the active-nucleus need to maintained as detectable).

Further, it is stressed that the second binding region (20, 21, 22, and 23 in FIGS. 1-6) in the targeting carrier comprises that portion of the subject biosensor that interacts with the target species/substrate/molecule/analyte. The first and second binding regions may be essentially identical, overlapping, or coextensive or separated by a plurality of atoms.

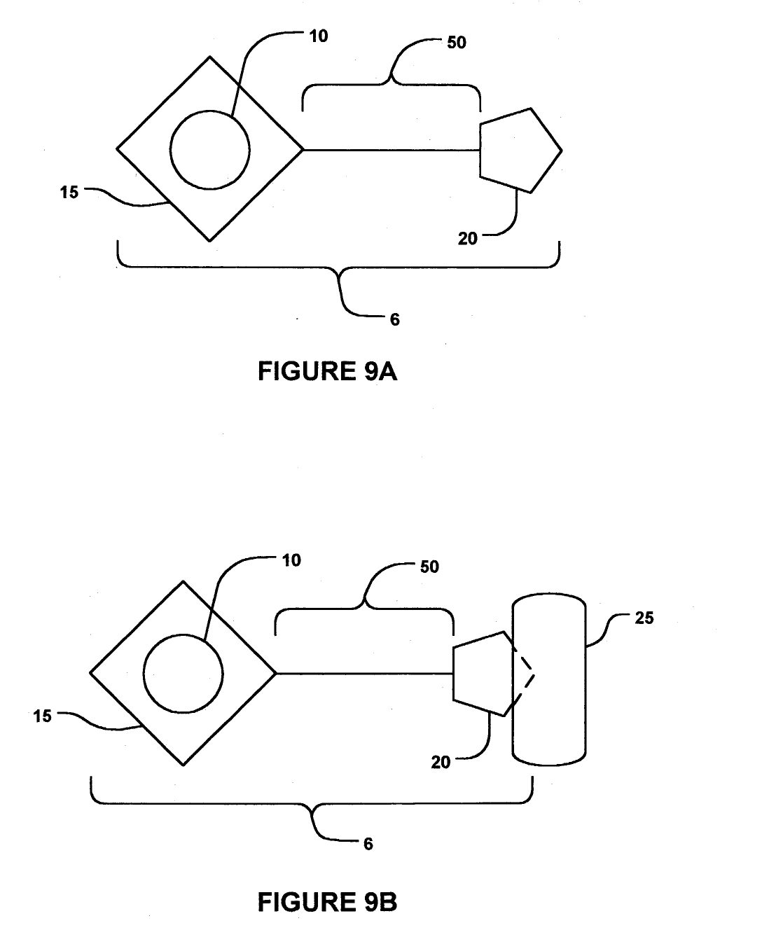

Clearly, the embodiments structures depicted in FIGS. 1-8 for the basic subject biosensor may contain additional useful components/structures. As seen in FIGS. 9A and 9B, one, or more. “tether” or “linker” or “spacer” regions 50 may be included in the biosensor 6. Specifically, FIG. 9A shows a biosensor comprising a first binding region 15 for the active-nucleus, a bound active-nucleus 10, a second binding region 20 for the target, and a tether 50. The tether 50 serves to separate the first 15 and second 20 binding regions and may serve as a site where chemical modification can occur. FIG. 9B illustrates the binding of the second binding group 20 with a target 25. The chemical nature of the tether may be varied and includes polymethylenes, homo and heteropolymers, polyethers, amides, various functional group combinations, amino acids, carbohydrates, and the like. If desired, a plurality of tethered second binding groups may be bound to a first binding region, with each tether and/or second binding group the same or different.

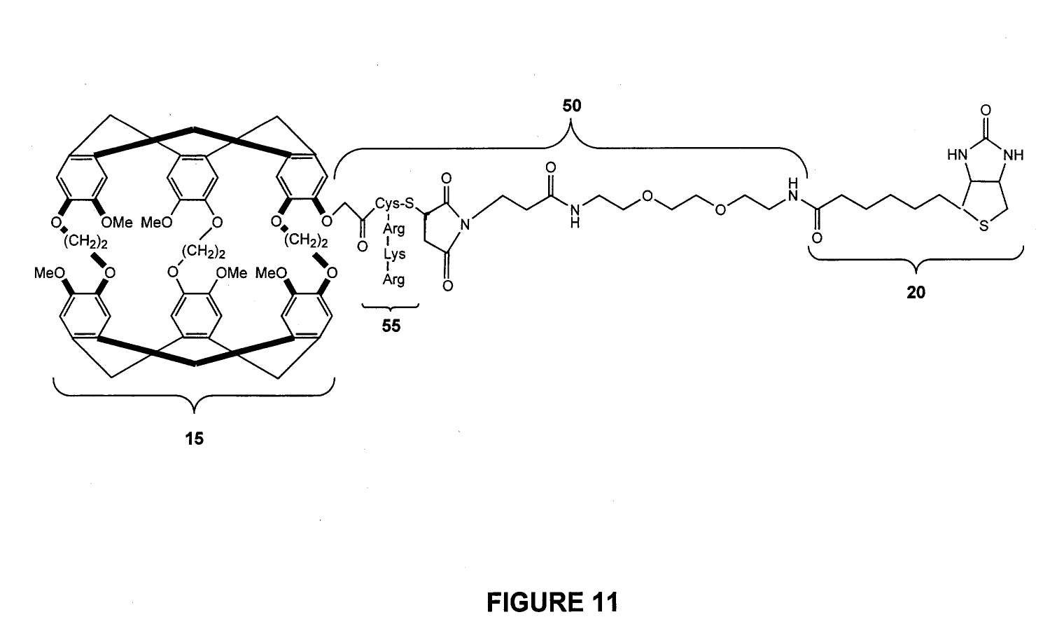

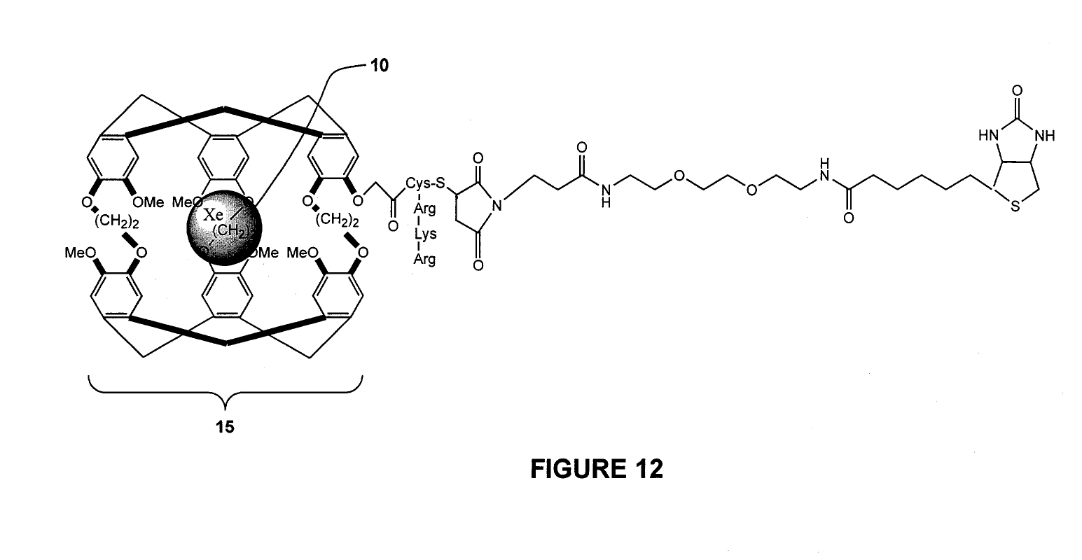

The tether may be derivatized to include a solubilizing region or other desired chemical feature such as additional binding sites and the like. The solubilizing region aids in solubilizing the biosensor in either a hydrophilic or hydrophobic environment. It is noted that a solubilizing region may also be included, either in addition to or separately, in the first and/or second binding regions. A water solubilizing region may include generally hydrophilic groups such as peptides, carbohydrates, alcohols, amines, and the like (for a specific example see FIGS. 11 and 12).

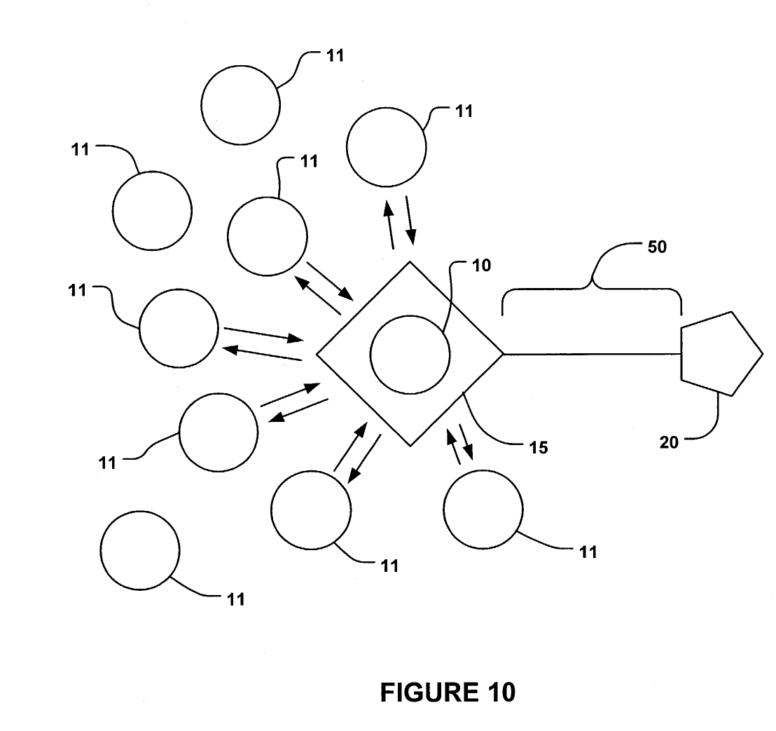

FIG. 10 illustrates a subject biosensor in which the signal is enhanced by a rapid chemical exchange of the active-nuclei. Free active-nuclei 11 rapidly exchange with the active-nucleus 10 bound in the first binding region 15 to produce an overall increase in sensitivity by accumulating the signal in the free state. More specifically, a functionalized active-nucleus biosensor is disclosed that capitalizes on the enhanced signal to noise, spectral simplicity, and chemical shift sensitivity of a hyperpolarized xenon to detect specific biomolecules at the level of tens of nanomoles. Optical pumping (T. G. Walker, W. Happer, Reviews of Modern Physics 69, 629-642 (1997)) has enhanced the use of xenon as a sensitive probe of its molecular environment (C. I. Ratcliffe, Annual reports on NMR spectroscopy 36, 124-208 (1998) and Y. Q. Song, B. M. Goodson, A. Pines, Spectroscopy 14, 26-33 (1999)). Laser-polarized xenon has been utilized as a diagnostic agent for medical magnetic resonance imaging (MRI) (M. S. Albert et al., Nature 370, 199-201 (1994)) and spectroscopy (J. Wolber, A. Cherubini, M. O. Leach, A. Bifone, Magnetic Resonance in Medicine 43, 491-496 (2000)), and as a probe for the investigation of surfaces and cavities in porous materials and biological systems. As indicated for an active-nucleus, xenon provides information both through direct observation of its NMR spectrum (C. R. Bowers et al., Journal of the American Chemical Society 121, 9370-9377 (1999), R. F. Tilton, I. D. Kuntz, Biochemistry 21, 6850-6857 (1982), M. A. Springuel-Huet, J. L. Bonardet, A. Gedeon, J. Fraissard, Magnetic Resonance in Chemistry 37, S1-S13 (1999), M. Luhmer et al., Journal of the American Chemical Society 121, 3502-3512 (1999), K. Bartik, M. Luhmer, J. P. Dutasta, A. Collet, J. Reisse, Journal of the American Chemical Society 120, 784-791 (1998), S. M. Rubin, M. M. Spence, B. M. Goodson, D. E. Wemmer, A. Pines, Proceedings of the National Academy of Sciences of the United States of America 97, 9472-9475 (2000), and T. Brotin, A. Lesage, L. Emsley, A. Collet, Journal of the American Chemical Society 122, 1171-1174 (2000)) and by the transfer of its enhanced polarization to surrounding spins (G. Navon et al., Science 271, 1848-1851 (1996) and C. Landon, P. Berthault, F. Vovelle, H. Desvaux, Protein Science 10, 762-770 (2001)). In a protein solution, weak xenon-protein interactions render the chemical shift of xenon dependent on the accessible protein surface, and even allow the monitoring of the protein conformation (Rubin, S. M. Spence, M. M., Dimitrov, I. E., Ruiz, E. J., Pines, A. and Wemmer, D., Journal of the American Chemical Society, 123, 8616-8617 (Aug. 7, 2001)). In order to utilize xenon as a specific sensor of target molecules the xenon was functionalized for the purpose of reporting specific interactions with the molecular target.

Specifically, a laser polarized xenon was “functionalized” by a biotin-modified supramolecular cage to detect biotin-avidin binding, thus, the specific target is avidin. Although, as previously indicated, the first binding region that holds the active-nucleus may be one of many possible structures, one suitable first binding region or cage is a member of the cryptophane family. Cryptophane has the following structure:

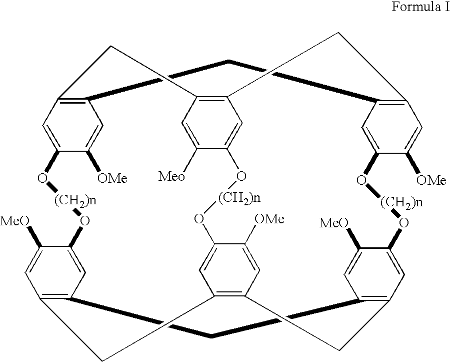

wherein n=2 for cryptophane-A or n=3 for cryptophane-E. It is stressed that other combinations of n are possible and include, but are not limited to n=2 or 3 and any combination thereof for other contemplated structures.

FIG. 11 (showing Formula II) depicts a specific targeting carrier in which the first binding region cryptophane-A 15 is covalently attached to a tether 50, having a solubilizing region 55, and biotin as the second binding region 20 (see the Example #1 below for synthesis details). The solubilizing region comprises a short peptide chain (Cys-Arg-Lys-Arg) having positively charged groups at physiological pH values.

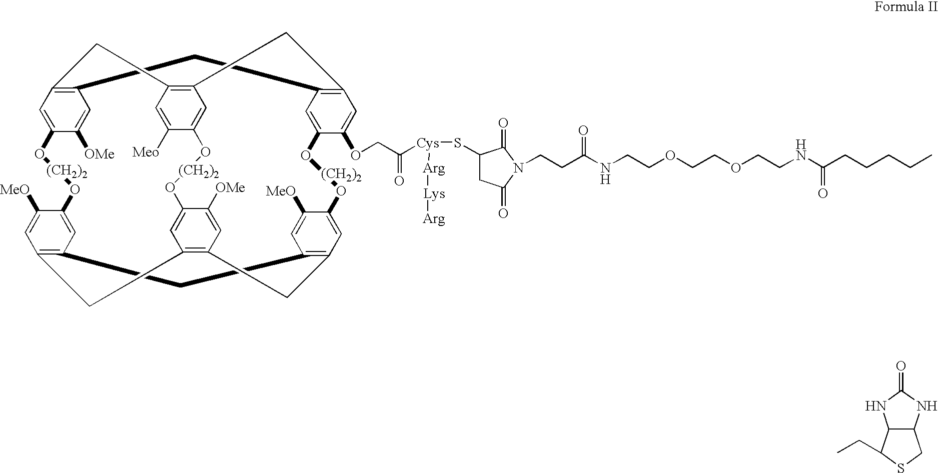

FIG. 12 (showing Formula III) shows the functionalized active-nucleus biosensor when the xenon 10 is bound within the first binding region cryptophane-A 15 cage.

//

//

//

//

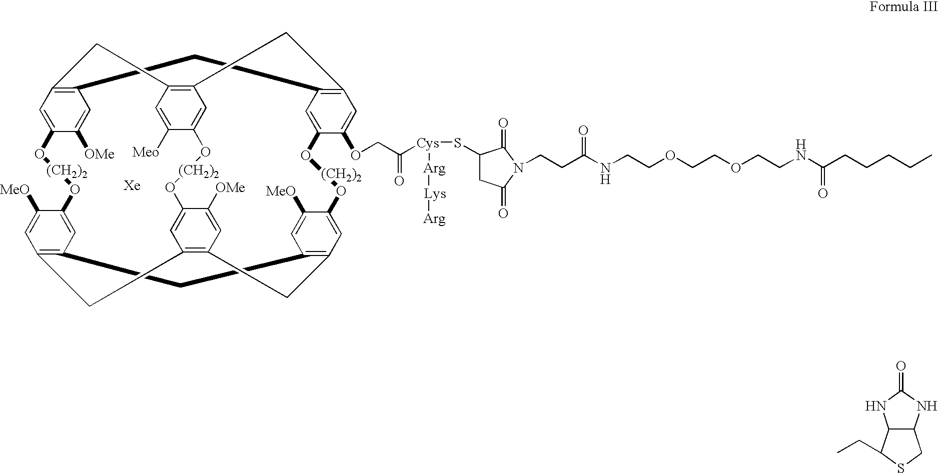

Non-Functionalized Active Nucleus Sensor

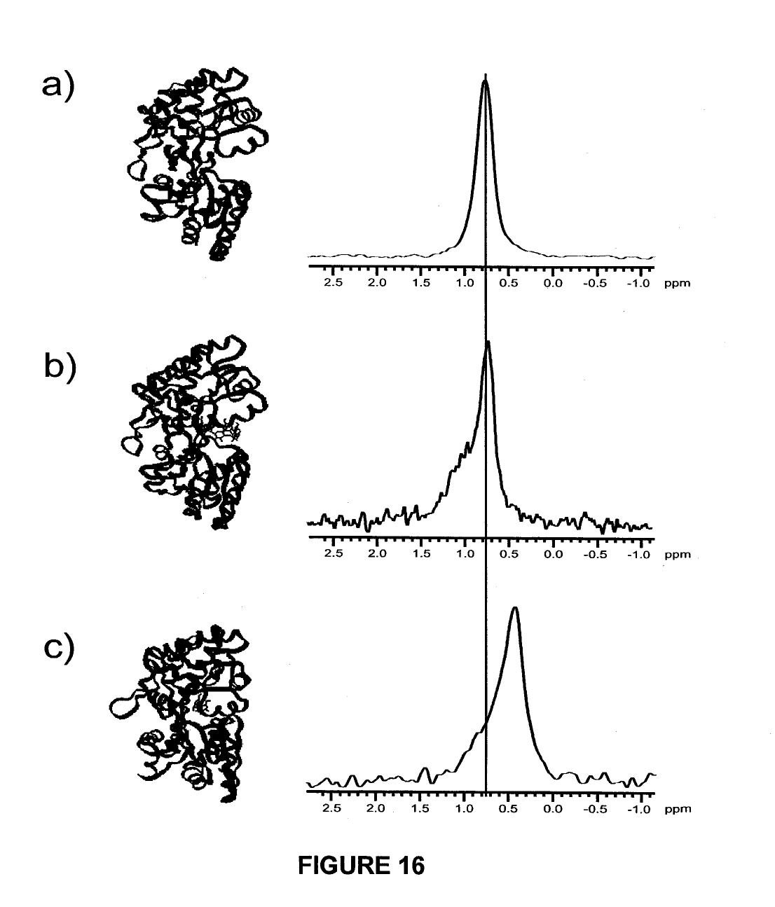

In another embodiment of the subject invention a non-functionalized active-nucleus sensor is disclosed and related, in its various aspects, depicted in FIGS. 16-28. To verify that a non-fuctionalized active-nucleus sensor, such as hyperpolarized ¹²⁹Xe, functions as a binding/conformational change sensor, several protein targets were selected as exemplary macromolecules, with no intention of limiting the targeted species to just proteins. The exemplary protein target were: maltose binding protein (MBP), ribose binding protein (RBP), glucose/galactose binding protein (GGBP), and nitrogen transcription regulator C (NTRC). In particular, MBP is a periplasmic protein in Gram-negative bacteria that plays a role in active transport and serves as an initial receptor for chemotaxis (Schwartz, M. In Escherichia Coli and Salmonella Typhimurium: Cellular and Molecular Biology; Neidhardt, F. C., Ingraham, J. L., Low, K. B., Magasanik, B., Schaechter, M., Umbarger, H. E., Eds.; American Society for Microbiology: Washington, D.C., (1987); Vol. 2, pp 1482-1502). MBP (MW˜41,700) binds maltose, other linear maltodextrins, and cyclodextrins with high affinity, but it binds glucose with low affnity (Szmelcman, S.; Schwartz, M.; Silhavy, T. J.; Boos, W., Eur. J. Biochem. (1976), 65, 13-19). The three-dimensional structures of MBP, both unliganded and complexed with maltose, have been determined by x-ray crystallography to 1.8 and 2.3 Å, respectively (Spurlino, J. C.; Lu, G. -Y.; Quiocho, F. A., J. Biol. Chem. (1991), 266, 5202-5219 and Sharff, A. J.; Rodseth, L. E.; Spurlino, J. C.; Quiocho, F. A., Biochemistry(1992), 31, 10657-10663). The sugar binding site is located in a cleft between the two domains of the protein; maltose binding (K˜9×10⁵ M⁻¹) induces a large structural change from an “open” unliganded conformer (FIGS. 16a and b) to a “closed” structure of the complex (FIG. 16c). The conformational change of MBP upon addition of maltose has been detected in solution by a number of physical measurements including fluorescence, electron paramagnetic resonance, small angle x-ray scattering, and ¹H NMR (Szmelcman, S.; Schwartz, M.; Silhavy, T. J.; Boos, W. Eur. J. Biochem. (1976), 65, 13-19, Shilton, B. H.; Flocco, M. M.; Nilsson, M.; Mowbray, S. L. J. Mol. Biol. (1996), 264, 350-363, Hall, J. A.; Gehring, K.; Nikaido, H. J. Biol. Chem. (1997), 272, 17695-17609, Hall, J. A.; Thorgeirsson, T. E.; Liu, J.; Shin, Y. -K.; Nikaido, H. J. Biol. Chem. (1997), 272, 17610-17614, and Gehring, K.; Zhang, X.; Hall, J. A.; Nikaido, H.; Wemmer, D. E. Biochem. Cell Biol. (1998), 76, 189-197). As is seen with MBP (see below and FIGS. 16-18), the sensitivity of the ¹²⁹Xe chemical shift to the conformation of MBP indicates that xenon is useful as a probe of protein functional states and ligand-protein binding interactions in solution. ¹²⁹Xe NMR has many advantages for use in a biochemical assay and the employment of laser-polarized xenon assures rapid acquisition of data. The fact that biomolecular states are reported through an independent, chemically inert species in solution alleviates the need for labeling molecules of interest with radioisotope or fluorescent probes and allows for full recovery of biomolecular samples in their native state. Furthermore, the detection of ¹²⁹Xe NMR signals from xenon in solution is facilitated by a simple spectrum with no background. The ability to detect protein conformational changes through the ¹²⁹Xe chemical shift should be quite general as is indicated by additional protein systems.

Rationale for Sensitivity of Non-Functionalized Active Nucleus Sensor

The affinity of xenon for hydrophobic cavities in macromolecular interiors (Schoenborn, B. C., Watson, H. C. & Kendrew, J. C. (1965), Binding of Xenon to Sperm Whale Myoglobin. Nature, 207, 28-30) has motivated a variety of applications of this inert gas in biochemical and structural studies of proteins. Xenon has been used to identify protein active sites and identify cavities that might be part of pathways by which substrates reach active sites (Montet, Y., Amara, P., Volbeda, A., Vernede, X., Hatchikian, E. C., Field, M. J., Frey, M. & Fontecilla-Camps, J. C. (1997), Gas Access to the Active Site of Ni—Fe Hydrogenases Probed by X-ray Crystallography and Molecular Dynamics. Nat. Struct. Bio., 4(7), 523-526, Wentworth Jr., P., Jones, L. H., Wentworth, A. D., Zhu, X., Larsen, N. A., Wilson, I. A., Xu, X., Goddard III, W. A., Janda, K. D., Eschenmoser, A. & Lerner, R. A. (2001), Antibody Catalysis of the Oxidation of Water. Science, 293, 1806-1811, and Whittington, D. A., Rosenzweig, A. C., Frederick, C. A. & Lippard, S. J. (2001), Xenon and Halogenated Alkanes Track Putative Substrate Binding Cavities in the Soluble Methane Monooxygenase Hydroxylase. Biochemistry, 40, 3476-3482). Because of its small size and large polarizability, xenon has been used as part of model systems in both theoretical and experimental studies of ligand-protein binding (Tilton, R. F., Singh, U. C., Weiner, S. J., Connolly, M. I., Kuntz, I. D., Kollman, P. A., Max, N. & Case, D. A. (1986), Computational Studies of the Interaction of Myoglobin and Xenon. J. Mol. Biol., 192(2), 443-456, Mann, G. & Hermans, J. (2000), Modeling Protein-Small Molecule Interactions: Structure and Thermodynamics of Noble Gases Binding in a Cavity in Mutant Phage T4 Lysozyme L99A. J. Mol. Biol., 302, 979-989, and Quillin, M. L., Breyer, W. A., Griswold, I. J. & Matthews, B. W. (2000), Size Versus Polarizability in Protein-Ligand Interactions: Binding of Noble Gases within Engineered Cavities in Phage T4 Lysozyme. J. Mol. Biol., 302, 955-977). A recent investigation demonstrated the ability of xenon to catalyze enzymatic reactions with radical pair intermediates (Anderson, M., Xu, Y. & Grissom, C. B. (2001), Electron Spin Catalysis by Xenon in an Enzyme. J. Am. Chem. Soc., 123, 6720-6721). In addition, there has been increasing use of xenon for determining phases in protein x-ray crystallography by both multiple isomorphous replacement (MIR) and multiwavelength anomalous diffraction (MAD) techniques (Schiltz, M., Prangé, T. & Fourme, R. (1994), On the Preparation and X-ray Data Collection of Isomorphous Derivatives. J. App. Cryst., 27, 950-960, Soltis, S. M., Stowell, M. H. B., Wiener, M. C., Phillips, G. N., Jr. & Rees, D.C. (1997), Successful Flash-Cooling of Xenon-Derivatized Myoglobin Crystals. J. Appl. Crystallogr., 30, 190-194, Ogata, C. M. (1998), MAD Phasing Grows Up. Nat. Struct. Biol., 5(8), 638-640, Owen, D. J. & Evans, P. R. (1998), A Structural Explanation for the Recognition of Tyrosine-Based Endocytotic Signals. Science, 282, 1327-1332, and Hamburger, Z. A., Brown, M. S., Isberg, R. R. & Bjorkman, P. J. (1999), Crystal Structure of Invasin: A Bacterial Integrin-Binding Protein. Science, 286, 291-295). Despite the numerous ways investigators can employ specific xenon-protein interactions, there exists no simple assay for xenon binding. Here we further develop the use of ¹²⁹Xe NMR to detect protein cavities that bind xenon and explore using ¹²⁹Xe as a reporter of cavity structure.

The sensitivity of the ¹²⁹Xe chemical shift to its local chemical environment (Ratcliffe, C. (1998), Xenon NMR. Annu. Rep. NMR Spectrosc., 36, 124-208) has motivated the use of xenon as an NMR probe of biomolecules (Miller, K. W., Reo, N. V., Schoot Uiterkamp, A. J., Stengle, D. P., Stengle, T. R. & Williamson, K. L. (1981), Xenon NMR: Chemical Shifts of a General Anesthetic in Common Solvents, Proteins, and Membranes. Proc. Natl. Acad. Sci. USA, 78(8), 4946-4949, Tilton, R. F., Jr. & Kuntz, I. D., Jr. (1982), Nuclear Magnetic Resonance Studies of Xenon-129 with Myoglobin and Hemoglobin. Biochemistry, 21(26), 6850-6857, and McKim, S. & Hinton, J. F. (1994), Evidence of Xenon Transport Through the Gramicidin Channel: a 129-Xe NMR Study. Biochim. Biophys. Acta., 1193(1), 186-198). Exploiting intense optically pumped ¹²⁹Xe NMR signals, (Raftery, D., Long, H., Meersman, T., Grandinetti, P. J., Reven, L. & Pines, A. (1991), High Field NMR of Adsorbed Xenon Polarized by Laser Pumping. Phys. Rev. Lett., 66, 584-586 and Walker, T. G. & Happer, W. (1997), Spin-Exchange Optical Pumping of Noble-Gas Nuclei. Rev. Mod. Phys., 69, 629-642) it has been possible to probe cavities in lyophilized lysozyme and lipoxygenase (Bowers, C. R., Storhaug, V., Webster, C. E., Bharatam, J., Cottone, A., Gianna, R., Betsey, K. & Gaffney, B. J. (1999), Exploring Surfaces and Cavities in Lipoxygenase and Other Proteins by Hyperpolarized Xenon-129 NMR. J. Am. Chem. Soc., 121(40), 9370-9377), detect blood oxygenation levels (Wolber, J., Cherubini, A., Dzik-Jurasz, A. S., Leach, M. 0. & Bifone, A. (1999), Spin-lattice Relaxation of Laser-Polarized Xenon in Human Blood. Proc. Natl. Acad. Sci. USA, 96(7), 3664-9), and identify ligand binding sites in a lipid transfer protein (Landon, C., Berthault, P., Vovelle, F. & Desvaux, H. (2001), Magnetization Transfer from Laser-Polarized Xenon to Protons in the Hydrophobic Cavity of the Wheat Nonspecific Lipid Transfer Protein. Prot. Sci., 10, 762-770) through the spin-polarization induced nuclear Overhauser effect (Navon, G., Song, Y. Q., Room, T., Appelt, S., Taylor, R. E. & Pines, A. (1996), Enhancement of Solution NMR and MRI with Laser-Polarized Xenon. Science, 271(5257), 1848-1851). With a functionalized cage, laser-polarized xenon was used to detect a ligand binding event (see this subject application and Spence, M. M., Rubin, S. M., Dimitrov, I. E., Ruiz, E. J., Wemmer, D. E., Pines, A., Yao, S. Q., Tian, F. & Schultz, P. G. (2001), Functionalized Xenon as a Biosensor. Proc. Nat. Acad. Sci. USA, 98(19), 10654-10657). The chemical shift of xenon in solution with the maltose binding protein (MBP) was shown to be sensitive to the conformation of the protein (see this subject application and Rubin, S. M., Spence, M. M., Dimitrov, I. E., Ruiz, E. J., Pines, A. & Wemmer, D. E. (2001), Detection of a Conformational Change in Maltose Binding Protein by ¹²⁹Xe NMR Spectroscopy. J. Am. Chem. Soc., 123, 8616-8617). Although these studies have demonstrated that the ¹²⁹Xe shift changes in different protein environments, the detailed mechanism for these changes remains elusive. Presented herein is a rationale for the ¹²⁹Xe chemical shift changes upon binding to hydrophobic cavities in proteins. By correlating NMR data with crystal structures, the influence of specific xenon-protein interactions on the ¹²⁹Xe shift is revealed. It is found that the presence of a xenon binding site can be deduced from ¹²⁹Xe chemical shift data alone and that the shift of xenon in such a site is affected by cavity structure.

¹²⁹Xe Chemical Shifts in Protein Solutions

Tilton and Kuntz first described the behavior of the ¹²⁹Xe NMR signal of xenon in solution with myoglobin (Tilton, R. F., Jr. & Kuntz, I. D., Jr. (1982), Nuclear Magnetic Resonance Studies of Xenon-129 with Myoglobin and Hemoglobin. Biochemistry, 21(26), 6850-6857). They observed a single resonance, reflecting fast exchange between xenon in water and xenon bound to a site in the protein that had been identified in previous crystal structures, and characterized the chemical shift as a weighted average of the shifts of xenon in these two environments. Nonspecific xenon-protein interactions were later shown to affect the ¹²⁹Xe shift in myoglobin solution (Rubin, S. M., Spence, M. M., Goodson, B. M., Wemmer, D. E. & Pines, A. (2000), Evidence of Nonspecific Surface Interactions Between Laser-Polarized Xenon and Myoglobin in Solution. Proc. Natl. Acad. Sci. USA, 97(17), 9472-9475 and Locci, E., Dehouck, Y., Casu, M., Saba, G., Lai, A., Luhmer, M., Reisse, J. & Bartik, K. (2001), Probing Proteins in Solution by ¹²⁹Xe NMR Spectroscopy. J. Magn. Reson., 150(2), 167-174), resulting in a complex pattern of up- and down-field shifts as a function of xenon and myoglobin concentrations. For all proteins studied there has only been a single ¹²⁹Xe resonance observed, with a chemical shift reflecting fast exchange of xenon among all specific and nonspecific binding sites (Bowers, C. R., Storhaug, V., Webster, C. E., Bharatam, J., Cottone, A., Gianna, R., Betsey, K. & Gaffney, B. J. (1999), Exploring Surfaces and Cavities in Lipoxygenase and Other Proteins by Hyperpolarized Xenon-129 NMR. J. Am. Chem. Soc., 121(40), 9370-9377, Rubin, S. M., Spence, M. M., Dimitrov, I. E., Ruiz, E. J., Pines, A. & Wemmer, D. E. (2001), Detection of a Conformational Change in Maltose Binding Protein by ¹²⁹Xe NMR Spectroscopy. J. Am. Chem. Soc., 123, 8616-8617, Locci, E., Dehouck, Y., Casu, M., Saba, G., Lai, A., Luhmer, M., Reisse, J. & Bartik, K. (2001),Probing Proteins in Solution by ¹²⁹Xe NMR Spectroscopy. J. Magn. Reson., 150(2),167-174, and Rubin, S. M., Spence, M. M., Pines, A. & Wemmer, D. E. (2001),Characterization of the Effects of Nonspecific Xenon-Protein Interactions on ¹²⁹Xe Chemical Shifts in Aqueous Solution: Further Development of Xenon as a Biomolecular Probe. J. Magn. Reson., 152(1), 79-86). Titration of ¹²⁹Xe solutions with amino acids, peptides, and denatured proteins results in a downfield shift, linear in solute concentration, that results from nonspecific interactions (Rubin, S. M., Spence, M. M., Pines, A. & Wemmer, D. E. (2001), Characterization of the Effects of Nonspecific Xenon-Protein Interactions on ¹²⁹Xe Chemical Shifts in Aqueous Solution: Further Development of Xenon as a Biomolecular Probe. J. Magn. Reson., 152(1), 79-86). The concentration-normalized change in chemical shift, denoted α and expressed in units of ppm/mM, depends on properties of the solute such as chemical functionality, charge, and size. The α values of denatured proteins increase linearly with the number of amino acids in the primary sequence with a slope approximately equal to 0.005 ppm/mM·amino acid.

With the exception of myoglobin, titrations of xenon solutions with native proteins have all resulted in downfield shifts in the ¹²⁹Xe signal that are linear with increasing protein concentration (Rubin, S. M., Spence, M. M., Dimitrov, I. E., Ruiz, E. J., Pines, A. & Wemmer, D. E. (2001), Detection of a Conformational Change in Maltose Binding Protein by ¹²⁹Xe NMR Spectroscopy. J. Am. Chem. Soc., 123, 8616-8617 and Rubin, S. M., Spence, M. M., Pines, A. & Wemmer, D. E. (2001), Characterization of the Effects of Nonspecific Xenon-Protein Interactions on ¹²⁹Xe Chemical Shifts in Aqueous Solution: Further Development of Xenon as a Biomolecular Probe. J. Magn. Reson., 152(1), 79-86). Despite the fact that unfolded proteins have more surface area exposed for nonspecific xenon interactions, the a values of several native proteins (e.g. bovine serum albumin and unliganded MBP) are greater than for their denatured forms. It was suggested that this discrepancy arises from specific binding interactions in the native proteins that contribute to the ¹²⁹Xe chemical shift (Rubin, S. M., Spence, M. M., Pines, A. & Wemmer, D. E. (2001), Characterization of the Effects of Nonspecific Xenon-Protein Interactions on ¹²⁹Xe Chemical Shifts in Aqueous Solution: Further Development of Xenon as a Biomolecular Probe. J. Magn. Reson., 152(1), 79-86). We report here the structure of MBP in the presence of xenon and confirm that, as anticipated by the α value, the protein binds xenon in an interior cavity. Changes in xenon affinity for this cavity upon addition of maltose can account for the sensitivity of the ¹²⁹Xe chemical shift to the conformation of MBP.

The fact that many specific and nonspecific interactions contribute to the α value of a native protein makes it difficult to determine the effects on the ¹²⁹Xe chemical shift of particular specific binding sites. We examined the effects of a single interaction by comparing a values for T4 lysozyme with a xenon binding site created or blocked by mutation or competitive binding. In this case, all xenon-protein interactions remain the same except at the cavity of interest. This approach follows the work of Tilton and Kuntz, who observed changes in the ¹²⁹Xe shift in myoglobin solution upon addition Hgl⁻³, which blocks xenon binding (Tilton, R. F., Jr. & Kuntz, I. D., Jr. (1982), Nuclear Magnetic Resonance Studies of Xenon-129 with Myoglobin and Hemoglobin. Biochemistry, 21(26), 6850-6857). The set of T4 lysozyme mutants described by Matthews and coworkers is an ideal system for such mutation and binding inhibition studies (Eriksson, A. E., Baase, W. A., Zhang, X.-J., Heinz, D. W., Blaber, M., Baldwin, E. P. & Matthews, B. W. (1992), Response of a Protein Structure to Cavity-Creating Mutations and Its Relation to the Hydrophobic Effect. Science, 255, 178-183). Cavities created by mutations of bulky hydrophobic residues bind xenon, and the structures of these complexes have been determined by x-ray crystallography (Quillin, M. L., Breyer, W. A., Griswold, I. J. & Matthews, B. W. (2000), Size Versus Polarizability in Protein-Ligand Interactions: Binding of Noble Gases within Engineered Cavities in Phage T4 Lysozyme. J. Mol. Biol., 302, 955-977). By comparing the α values of these different proteins in the presence and absence of xenon inhibitors, the effects of the various cavity structures on the ¹²⁹Xe chemical shift can be elucidated.

### EXPERIMENTAL EXAMPLE #1

### Functionalized Xenon as a Biosensor

By way of example and not by way of limitation, one embodiment of the subject invention comprises a functionalized system that exhibits molecular target recognition. FIG. 11 (without xenon) and 12 (with xenon) show a biosensor molecule designed to bind both xenon and protein. Analogous to the general schematic diagrams seen in FIGS. 9A and 9B, the specifically synthesized subject biosensor molecule consists of three parts: the cage 15, which contains the xenon 10; the ligand 20, which directs the functionalized xenon 10 to a specific protein; and the tether 50, which links the ligand 20 and the cage 15. In this molecule, it is expected that the binding of the ligand 20 to the target protein (as in analogous FIG. 9B) will be reflected in a change of the xenon NMR spectrum.

The biotin (ligand second binding region 20) and avidin (target species) couple was chosen because of its high association constant (˜10¹⁵ M⁻¹) (P. C. Weber, D. H. Ohlendorf, J. J. Wendoloski, F. R. Salemme, Science 243, 85-88 (1989)) and the extensive literature characterizing binding properties of modified avidin or biotin (M. Wilchek, E. A. Bayer, Methods in Enzymology 184, 14-45 (1990)). The cage 15 chosen for this embodiment was a cryptophane-A molecule (A. Collet, Tetrahedron 43, 5725-5759 (1987)) with a polar peptide chain (solubilizing region 55) attached in order to make the cryptophane-A water-soluble.

The cryptophane-A-based biosensor molecule was synthesized by a modified template directed procedure (A. Collet, Tetrahedron 43, 5725-5759 (1987)). Starting from 3,4-dihydroxybenxaldehyde and using allyl bromide to reversibly protect the meta-hydroxyl group (S. N. Kilenyi, J. M. Mahaux, E. Vandurme, Journal of Organic Chemistry 56, 2591-2594 (1991)), one of the 6 methoxyl groups in cryptophane-A was regioselectively replaced with a free hydroxyl group. Upon reacting with methyl bromoacetate followed by hydrolysis (J. Canceill, A. Collet, G. Gottarelli, P. Palmieri, Journal of the American Chemical Society 109, 6454-6464 (1987)), the hydroxyl group in the modified cryptophane-A was converted to a carboxylic acid, which was subsequently coupled (using HOBt/HBTU/DIEA activation method) to the amino-terminus of a protected short peptide CysArgLysArg on rink amide resin. The resulting cryptophane-A-peptide conjugate was deprotected and cleaved off the resin using “Reagent K” (D. S. King, C. G. Fields, G. B. Fields, International Journal of Peptide and Protein Research 36, 255-266 (1990)), followed by purification with RP-HPLC (Microsorb™ 80210C5, RP-C18 column, flow 4.5 ml/min, buffer A: 0.1% TFA inH₂O, buffer B: 0.1% TFA in CH₃CN, linear gradient from 40% to 80% buffer B in 30 min). The purified conjugate was reacted with EZ-link TMPEO-Maleimide activated biotin (Pierce) to give the desired functionalized water-soluble cryptophane-A, which was further purified by RP-HPLC (same conditions). The last two peptide conjugated-products were verified by matrix-assisted laser desorption/ionization (MALDI)-time of flight-(TOF) mass spectrometry. All other intermediates were confirmed by ¹H NMR and MALDI-Fourier Transform mass spectrometry (FTMS).

Cryptophane-A has been shown to bind xenon with a binding constant K≈10³ M⁻¹ in organic solvents (K. Bartik, M. Luhmer, J. P. Dutasta, A. Collet, J. Reisse, Journal of the American Chemical Society 120, 784-791 (1998)) but the affinity is likely to increase in aqueous solution because of the hydrophobic nature of xenon. The characteristic chemical shift for xenon inside a cryptophane-A molecule is very unusual for xenon dissolved in solution, approximately 130 ppm upfield from that of xenon in water. The only background xenon signal in the sample arises from xenon free in solvent, so the signal from the functionalized xenon is easily distinguishable. In the design of a xenon biosensor, a separate peak corresponding to xenon encapsulated by the cage is necessary, requiring both strong binding and a large difference between the xenon chemical shifts in the cage and solvent environments. The spin-lattice relaxation time for the functionalized xenon described herein was measured to be greater than 40 s, sufficient time for the required transfer, mixing, and detection of the polarized xenon.

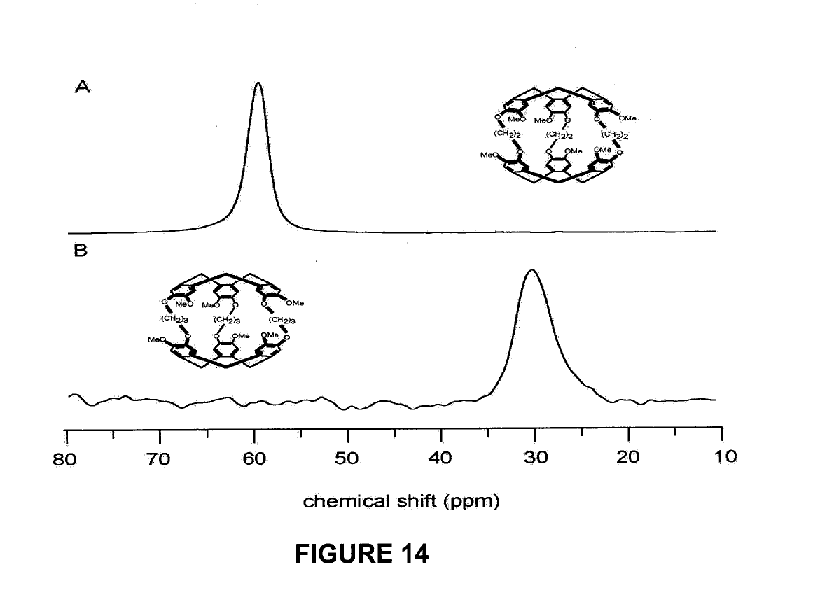

The functionalized biosensor solution was prepared by dissolving ˜0.5 mg of the cryptophane derivative (M.W.=2008 g mol⁻¹) in 700 μL of D₂O, yielding a concentration of ˜300 μM. This concentration was consistent with absorbance measurements at 284 nm (ε284=36,000 M⁻¹ cm⁻¹, determined for unmodified cryptophane-A by successive dilutions of a solution of known concentration). Approximately 80 nmol of affinity purified egg white avidin (Sigma) was used without further purification. Only half of the sample was located inside the detection region, so spectra actually reflect detection of ˜40 nmol avidin monomer. Natural abundance xenon (Isotec) was polarized and introduced to the sample using previously described methods (S. M. Rubin, M. M. Spence, B. M. Goodson, D. E. Wemmer, A. Pines, Proceedings of the National Academy of Sciences of the United States of America 97, 9472-9475 (2000)), showing ˜5% polarization for the spectra shown in FIGS. 13 and 14. All NMR spectra displayed were obtained in single acquisition experiments at a nominal ¹²⁹Xe frequency of 82.981 MHz.

FIG. 13 shows the full ¹²⁹Xe NMR spectrum of the functionalized xenon in the absence of protein (the trace running near the bottom axis and having a far left peak and far right peaks). The far left peak at 193 ppm corresponds to xenon free in water while the far right peaks around 70 ppm are associated with xenon-bound cryptophane-A. The far right peaks are shown expanded in the center of FIG. 13, where the more intense, upfield peak (˜70.7 ppm) corresponds to functionalized xenon and has a linewidth of 0.15 ppm (shown by the generalized schematic model, as seen in FIG. 9A). A smaller, middle peak (˜71.5 ppm) approximately 1 ppm downfield of the functionalized xenon peak is attributed to xenon bound to a bare cage, without linker and ligand. As the unfunctionalized caged xenon does not interact specifically with the protein, it serves as a useful reference for the chemical shift and signal intensity of the functionalized xenon in the binding event.

Upon addition of ˜80 nmol of avidin monomer, a third peak (˜73 ppm) appears approximately 2.3 ppm downfield of the functionalized xenon peak, attributable to functionalized xenon bound to the protein. Correspondingly, the peak assigned to free functionalized xenon decreases in intensity relative to the reference peak while its position remains unchanged. The peak (˜73 ppm) observed upon the addition of avidin is an unambiguous identifier of biotin-avidin binding, and hence the presence of avidin in solution.

The mechanism of the chemical shift change upon binding may result from actual contact between the cryptophane cage and the protein, leading to cage deformation and distortion of the xenon electron cloud. Changes in the rotational and vibrational motions of the cryptophane cage caused by binding to the protein could also affect the xenon chemical shift. Indeed, the sensitivity of xenon to perturbations of the first binding region cage is so great that deuteration of one methyl group results in a readily discernible change in the bound xenon chemical shift (T. Brotin, A. Lesage, L. Emsley, A. Collet, Journal of the American Chemical Society 122, 1171-1174 (2000)).

The subject methodology described herein offers the capability of multiplexing by attaching different second binding regions ligands to different first binding region cages, forming xenon sensors associated with distinct, resolved chemical shifts. As an example of this feature of the subject invention, FIG. 14 shows the changes in bound xenon chemical shift caused by using two different first binding region cages. The top spectrum A is that of xenon bound to cryptophane-A (n=2 in Formula 1 above) in a tetrachloroethane solution and the lower spectrum B is that of xenon bound to cryptophane-E (n=3 in Formula 1 above), similar to cryptophane-A, but with an additional methylene group added to each of the bridges between the caps. The resulting bound xenon chemical shift is ˜30 ppm upfield from that of xenon bound to cryptophane-A. The linewidths for cryptophanes A and E are broadened by the exchange of xenon between the cage and tetrachloroethane, the organic solvent used.

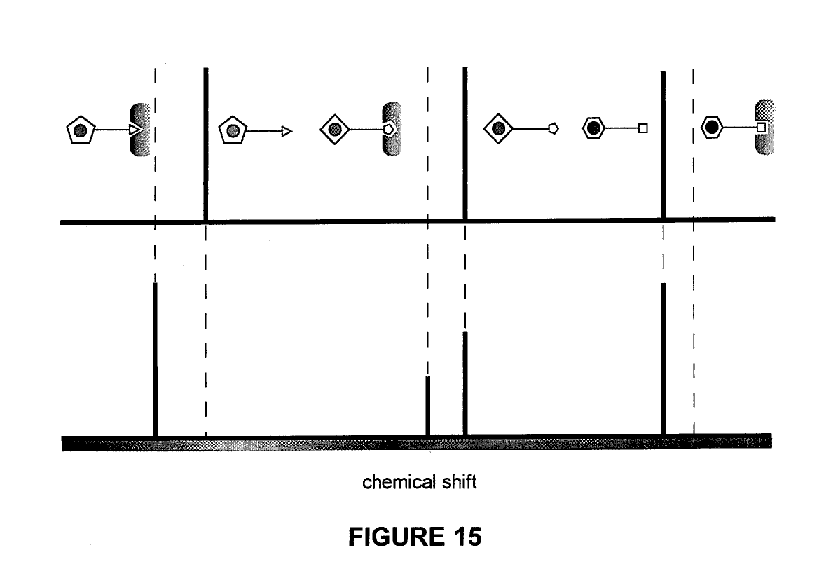

The diagram in FIG. 15 indicates schematically a multiplexing system (multiple functionalized xenon biosensors) for protein assay or screening procedures. The binding event assay/screening procedures would be distributed over a large chemical shift range by attaching each second binding region ligand to a different first binding region cage. In the absence of the targeted proteins, the spectrum, depicted in FIG. 15, would consist of three resolved xenon resonances because of the effect on the xenon chemical shift caused by cage modifications. Upon binding each of the targeted proteins, the xenon peaks should shift “independently,” signaling each binding event and reporting the existence of and amount of protein present. As long as the differences in shift between xenon in the different cages exceed the shift change upon binding, it should be possible to monitor and assign multiple binding events. In FIG. 15, the top spectrum shows the three distinct functionalized xenon peaks, corresponding to different cages linked to three ligands. The bottom spectrum shows the effect of adding the functionalized xenon to an unknown solution. Upon addition to the unknown solution, the leftmost peak shifts entirely, representing the case in which all functionalized xenon is bound to its corresponding protein. The central peak decreases in intensity and a peak corresponding to the protein-bound functionalized xenon appears. The rightmost peak remains unaffected, indicating the absence of the corresponding protein target.

Thus, enabling experimental data for the subject functionalized active-nucleus biosensor has been disclosed that exploits the chemical shift of functionalized xenon upon binding to a target species/substrate/molecule/analyte. The approach has several critical advantages over aspects of current biosensors, in that multiplexing assays and both heterogeneous and homogenous assays are possible. Furthermore, this methodology can be performed in biological materials in vitro or in vivo by combining the spatial encoding capabilities of MRI with the biosensing NMR capabilities of the functionalized xenon sensor. As indicated above, potential targets include, are not limited to, metabolites, proteins, toxins, nucleic acids, and protein plaques. It must be stated that, given the basic information presented herein, refinements of the subject functionalized detector molecules/sensors and the NMR procedures disclosed herein should further enhance the presented sensitivity by orders of magnitude, relative to the experimental example described herein and are within the realm of this disclosure.

### EXPERIMENTAL EXAMPLE #2

### Detection of Conformational Changes in Proteins Having a Known Xenon Binding Site with a Non-Functionalized ¹²⁹Xe Sensor: Maltose Binding Protein (MBP), Ribose Binding Protein (RBP), and Glucose/Galactose Binding Protein (GGBP)

A: Maltose Binding Protein (MBP)

MBP (SwissProt data base number MBP: P02925) was expressed from a PET vector in E. Coli BL21(DE3) cells and purified using DEAE ion-exchange and Superdex 75 size-exclusion chromatography (Pharmacia Biotech). Lyophilized protein was dissolved in a buffer containing 50 mM Tris-HCl pH 7.6, 100 mM KCl, 20% D₂O, and 1 mM sugar when indicated. In a manner similar to that previously reported,(Wolber, J.;

Cherubini, A.; Dzik-Jurasz, A. S.; Leach, M. 0.; Bifone, A. Proc. Natl. Acad. Sci. U.S.A. 1999, 96, 3664-3669) protein samples were mixed in a 2:1 ratio with buffer containing ˜4-5 mM laser-polarized xenon (natural abundance ¹²⁹Xe, Isotec) immediately prior to data acquisition. ¹²⁹Xe NMR spectra were acquired on a 300 MHz Varian Inova spectrometer using a single 90 pulse and a xenon polarization ˜1-5%. Sample concentrations were determined by absorbance at 280 nm (ε₂₈₀=66,350 M⁻¹cm⁻¹) after the acquisition of NMR data. Chemical shifts were determined from the highest point of each resonance peak with an estimated accuracy of 0.01 ppm.

FIG. 16 shows a ¹²⁹Xe spectra of laser-polarized xenon dissolved in solution containing 350 μM maltose binding protein (MBP) in the absence of ligand (a) and in the presence of 1 mM β-cyclodextrin (b), and 1 mM maltose (c). MBP undergoes a large conformational change only upon binding maltose, indicating that the difference of 0.35 ppm between the ¹²⁹Xe resonances is associated with the conformational state of MBP. Each spectrum is referenced to the ¹²⁹Xe chemical shift of xenon in buffer containing the corresponding sugar.

The effect of each sugar on the ¹²⁹Xe chemical shift is relatively small (0.01-0.04 ppm/mM); nevertheless, the reported ¹²⁹Xe shifts are referenced to the shift of xenon in buffer containing 1 mM sugar (see FIG. 16). The asymmetry of the peaks in frames “b” and “c” of FIG. 16 is likely an artifact that results from field inhomogeneities after rapid mixing of samples prior to acquisition; further refinement of the mixing process should improve the consistency of the peak shapes.

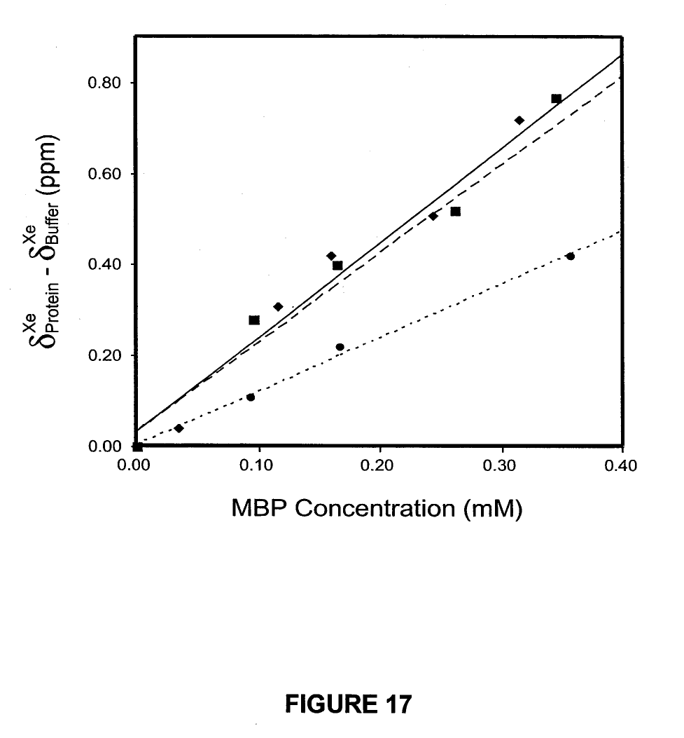

In order to characterize further the effect of MBP conformation on the ¹²⁹Xe chemical shift, shift measurements were made on a series of MBP solutions at varying protein concentration in the absence and presence of maltose; the results for the titrations are plotted in FIG. 17. The single resonance observed in each spectrum indicates that xenon is in fast exchange between the buffer and protein interaction sites. Analogous behavior has been observed, with traditionally techniques, for a number of proteins (Miller, K. W.; Reo, N. V.; Schoot Uiterkamp, A. J.; Stengle, D. P.; Stengle, T. R.; Williamson, K. L. Proc. Natl. Acad. Sci. U.S.A. 1981, 78, 4946-4949, Tilton, R. F., Jr.; Kuntz, I. D., Jr. Biochemistry 1982, 21, 6850-6857, McKim, S.; Hinton, J. F. Biochim. Biophys. Acta 1994, 1193, 186-198, Bowers, C. R.; Storhaug, V.; Webster, C. E.; Bharatam, J.; Cottone, A.; Gianna, R.; Betsey, K.; Gaffney, B. J. J. Am. Chem. Soc. 1999, 121, 1, 9370-9377, Rubin, S. M.; Spence, M. M.; Goodson, B. M.; Wemmer, D. E.; Pines, A. Proc. Natl. Acad. Sci. U.S.A. 2000, 97, 9472-9475, Locci, E.; Casu, M.; Saba, G.; Lai, A.; Luhmer, M.; Reisse, J.; Bartik, K. Private communication, and Rubin, S. M., Spence, M. M. Pines, A. and Wemmer, D. E. (2001) J. Magn. Reson., 152(1), 79-86, which is specifically incorporated herein by reference and is cited in Provisional Application No. 60/399,041). The observed shift is the average of the ¹²⁹Xe chemical shift in each environment weighted by the occupancy of xenon in that environment. As described previously, these environments can all be treated as weak binding sites that correspond either to diffusion-mediated nonspecific interactions between xenon and the protein surface or to specific xenon binding sites in the protein.

FIG. 17 shows ¹²⁹Xe chemical shift data as a function of MBP concentration in solution containing buffer alone (▪) and buffer with 1 mM β-cyclodextrin (♦), and 1 mM maltose (●). As observed in FIG. 17, the overall chemical shift changes linearly with protein concentration in the limit where a small fraction of xenon is bound to any site. The concentration-normalized shift, α (units of ppm/mM), has a characteristic value for a protein that depends on the number, strength, and effect on the ¹²⁹Xe shift of all the nonspecific and specific interactions of xenon with that protein (Rubin, S. M.; Spence, M. M.; Goodson, B. M.; Wemmer, D. E.; Pines, A. Proc. Natl. Acad. Sci. U.S.A. 2000, 97, 9472-9475 and Rubin, S. M., Spence, M. M. Pines, A. and Wemmer, D. E. (2001) J. Magn. Reson., 152(1), 79-86). A linear fit of the data in FIG. 17 yielded an α value for the unliganded, open conformer of MBP (α=2.1±0.2 ppm/mM) that is measurably greater than that of the closed MBP-maltose complex (α=1.2±0.1 ppm/mM).

A likely explanation for the difference in a values of the two conformers of MBP results is distinct specific xenon-protein interactions. For example, xenon binding sites may be unique to a particular conformer or the affinity of an identical site may change with protein conformation. The difference in α for two protein conformers may result solely from a change in the chemical character or area of the protein surface accessible to xenon. However, the change in the α value of MBP upon changing its conformation (Δα=0.9 ppm/mM) is likely too large to be accounted for by changes in nonspecific interactions alone (Rubin, S. M.; Spence, M. M.; Goodson, B. M.; Wemmer, D. E.; Pines, A. Proc. Natl. Acad. Sci. U.S.A. 2000, 97, 9472-9475 and Rubin, S. M., Spence, M. M. Pines, A. and Wemmer, D. E. (2001) J. Magn. Reson., 152(1),79-86). The α value of MBP complexed with β-cyclodextrin was measured to examine whether the sugar binding cleft itself was the site of such a specific interaction. Cyclodextrins bind MBP in the same cleft as maltose, but the larger size of the ligand hinders the closing of the two domains (Sharff, A. J.; Rodseth, L. E.; Quiocho, F. A. Biochemistry 1993, 32, 10553-10559). The similar α values of the open conformation (α=2.1±0.2 ppm/mM) and the MBP-β-cyclodextrin complex (α=2.0±0.1 ppm/mM) suggest that occluding the sugar binding site but maintaining the same overall structure does not significantly alter the xenon-protein interactions that give rise to the ¹²⁹Xe shift.

Changes in the ¹²⁹Xe shift that mark a conformational change due to ligand binding are detected in the presence of a large number of other species in solution; this phenomenon is possible because all contributions to the shift (in the limit of weak xenon binding) are additive.

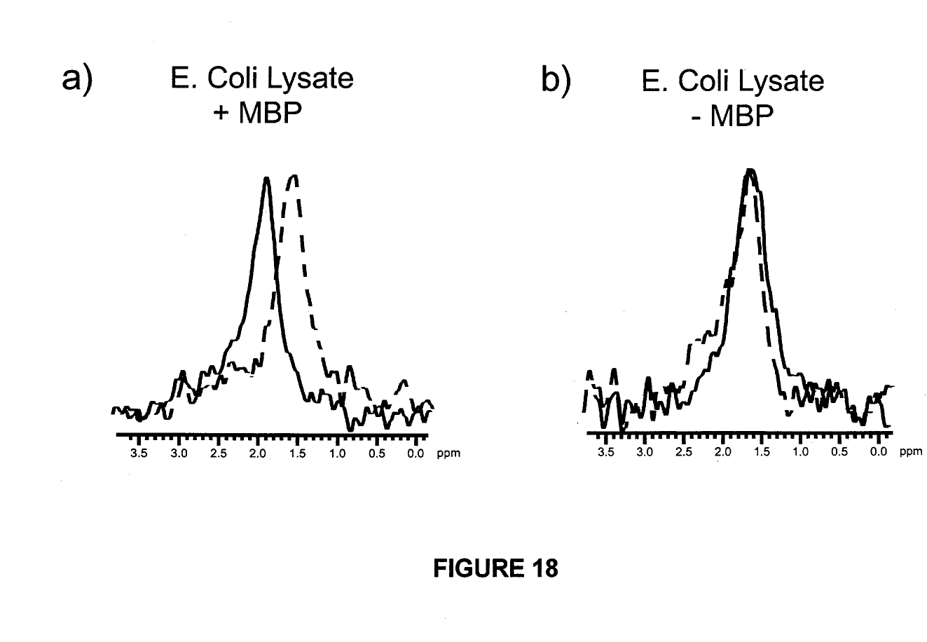

FIG. 18 shows ¹²⁹Xe NMR spectra of laser-polarized xenon dissolved in the crude cell lysates from two samples of E. coli with (a) and without (b) the expression plasmid for MBP. Addition of 1 mM maltose (spectra with broken line) has an observable effect on the ¹²⁹Xe chemical shift only for xenon in the lysate with MBP; the change of 0.25 ppm arises from the change in MBP conformation upon ligand binding from the “open” to the “closed” form.

Lysate samples were made by suspending the washed cell paste of 1 L cultures in 5 mL of lysis buffer containing 50 mM Tris-HCl pH 7.6, 100 mM KCl, 1 mM DTT, 5 mM EDTA, and 20% D₂O. Cells were lysed by sonication and spun at ˜50,000 g for 30 min. The resulting supernatant was used to make 0.5 mL samples for data acquisition; where indicated, concentrated maltose was added to a final concentration of 1 mM. MBP concentrations in the lysates from cells containing the plasmid for overexpression were estimated to be ˜200-300 μM by comparison with purified MBP on an SDS-polyacrylamide gel.

B: Ribose Binding Protein (RBP)

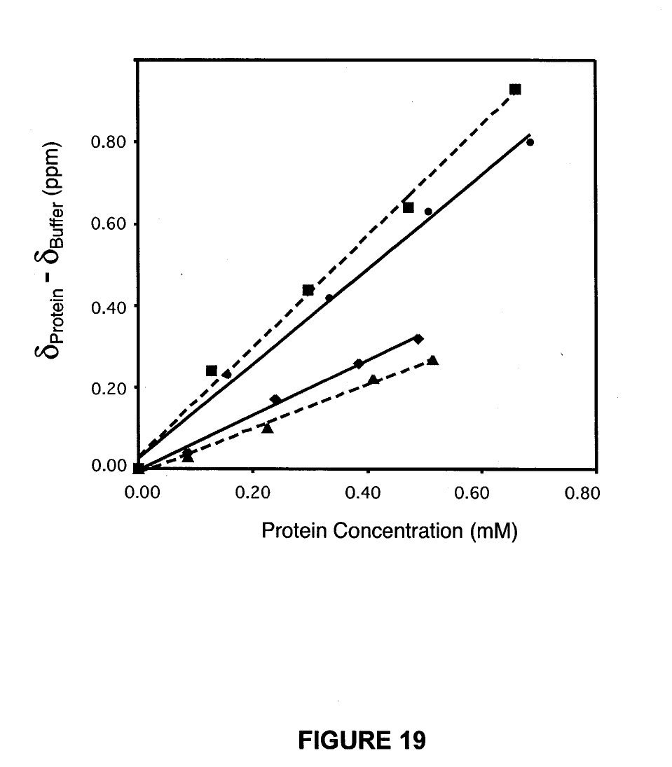

FIG. 19 shows ¹²⁹Xe chemical shift data for ribose binding protein (RBP) as a function of RBP concentration in solution containing buffer alone (♦) and buffer with 1 mM ribose (▴).

For RBP, the SwissProt protein data base number is RBP: P02925. RBP is from E. Coli and was expressed recombinantly in E. Coli. Expression of the gene was done from PET vector in E. Coli strain BL21 (DE3). Cells were grown in Luria Broth (LB) media to A₆₀₀=0.8 and induced with 1 mM IPTG. Cells were harvested three hours later. The RBP protein was purified with appropriate ion exchange and size exclusion chromatography. Buffer conditions for the NMR experiments are identical to that for MBP and the NMR data was collected in an equivalent manner to that utilized for MBP. Plainly, conformational changes/binding are observed in this experiment.

C: Glucose/Galactose Binding Protein (GGBP)

FIG. 19 also shows glucose/galactose binding protein (GGBP) as a function of GGBP concentration in solution containing buffer alone (●) and buffer with 1 mM glucose (▪).

For GGBP, the SwissProt protein data base number is GGBP: P02927. GGBP is from E. Coli and was expressed recombinantly in E. Coli. Expression of the gene was done from PET vector in E. Coli strain BL21 (DE3). Cells were grown in Luria Broth (LB) media to A₆₀₀=0.8 and induced with 1 mM IPTG. Cells were harvested three hours later. The GGBP protein was purified with appropriate ion exchange and size exclusion chromatography. Buffer conditions for the NMR experiments are identical to that for MBP and the NMR data was collected in an equivalent manner to that utilized for MBP. Clearly, conformational changes/binding are observed in this experiment.

### EXPERIMENTAL EXAMPLE #3

### Detection of Conformational Changes in a Protein with No Currently Known Xenon Binding Site With A Non-Functionalized ¹²⁹Xe Sensor: Nitrogen Transcription Regulator C (NTRC)

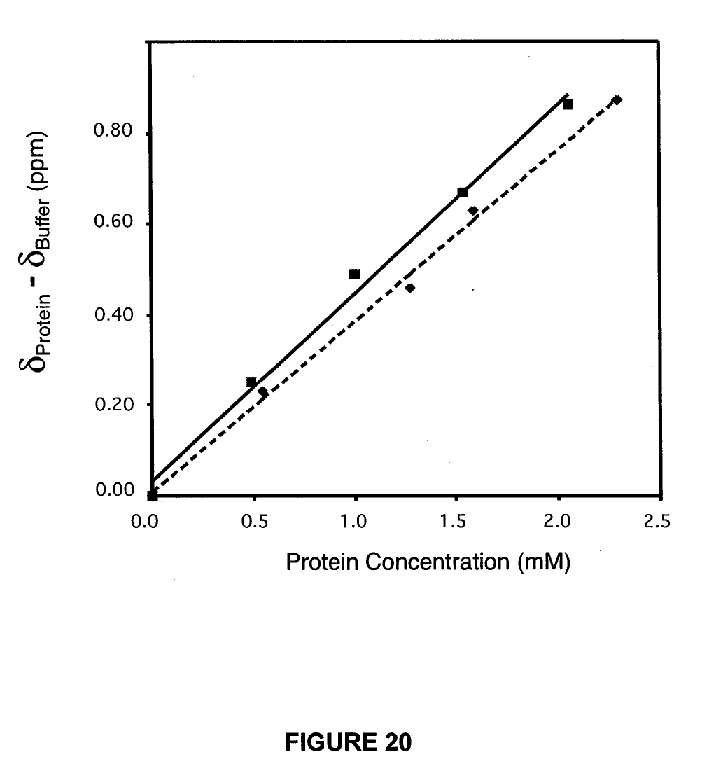

FIG. 20 shows ¹²⁹Xe chemical shift data for nitrogen transcription regulator (NTRC) as a function of NTRC concentration for the activated (NTRC BeFₓ) form (▪) and the inactivated form (♦).

For NTRC, the SwissProt protein data base number is NTRC: P41789. NTRC is from Salmonella Typhimurium and was expressed recombinantly in E. Coli. Expression of the gene was done from PET vector in E. Coli strain BL21 (DE3). Cells were grown in Luria Broth (LB) media to A₆₀₀=0.8 and induced with 1 mM IPTG. Cells were harvested three hours later. The NTRC protein was purified with appropriate ion exchange and size exclusion chromatography. Buffer conditions for the NMR experiments are identical to that for MBP and the NMR data was collected in an equivalent manner to that utilized for MBP. Conformational macromolecular changes/binding are visible in this experiment.

### EXPERIMENTAL EXAMPLE #4

### Support for Rationale for Sensitivity of Non-Functionalized Active Nucleus Sensor

Discussion of Results:

¹²⁹Xe Chemical Shifts of Xenon in MBP Solution

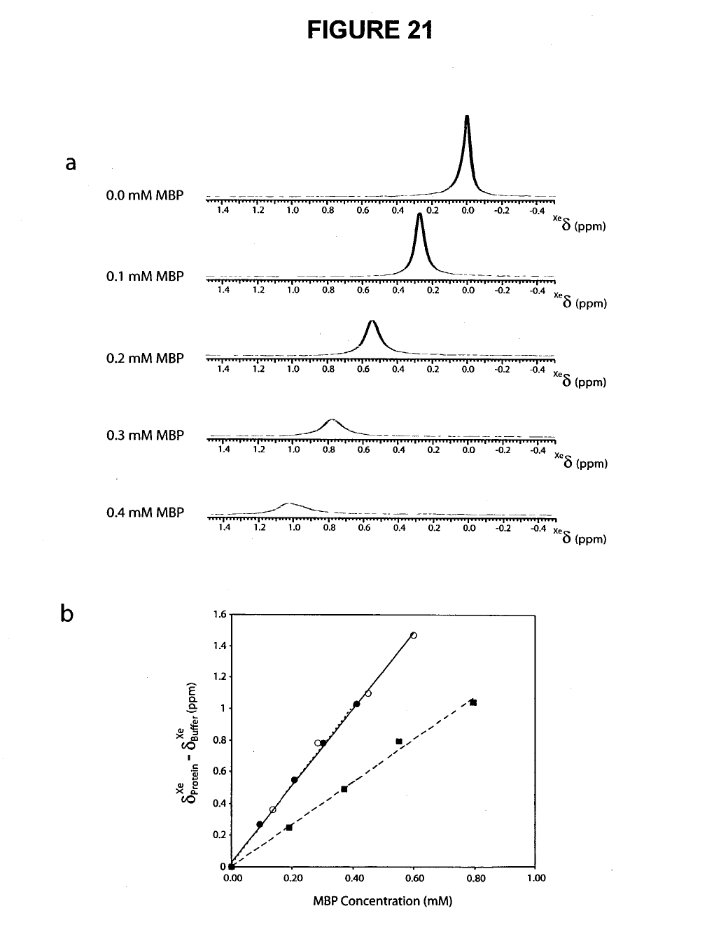

Addition of buffer with laser-polarized xenon to protein solutions enables rapid acquisition of ¹²⁹Xe NMR spectra for chemical shift determination. FIG. 21 shows a series of spectra of 1 mM xenon dissolved in solution with maltose binding protein at varying concentrations. Each spectrum is from a single acquisition with a total time for data collection of less than 5 minutes. The xenon line width increases with protein concentration. This broadening has been observed in other protein solutions and results from exchange of xenon between sites in the protein (both nonspecific and specific) and the solvent (Tilton, R. F., Jr. & Kuntz, I. D., Jr. (1982), Nuclear Magnetic Resonance Studies of Xenon-129 with Myoglobin and Hemoglobin. Biochemistry, 21(26), 6850-6857, Locci, E., Dehouck, Y., Casu, M., Saba, G., Lai, A., Luhmer, M., Reisse, J. & Bartik, K. (2001), Probing Proteins in Solution by ¹²⁹Xe NMR Spectroscopy. J. Magn. Reson., 150(2),167-174, and Rubin, S. M., Spence, M. M., Pines, A. & Wemmer, D. E. (2001), Characterization of the Effects of Nonspecific Xenon-Protein Interactions on ¹²⁹Xe Chemical Shifts in Aqueous Solution: Further Development of Xenon as a Biomolecular Probe. J. Magn. Reson., 152(1), 79-86). The decrease in signal with increasing protein concentration is due to increased spin-lattice relaxation of the xenon through protein protons, causing a loss of polarization during the time required to place the sample in the probe for data acquisition. At these concentrations of MBP and xenon, the resonance line width is modest (less than 0.2 ppm), which allows for measurement of the chemical shift with an estimated error of <0.02 ppm. As seen in the spectra and FIG. 21b, increasing protein concentrations shift the ¹²⁹Xe resonance downfield relative to xenon in buffer; the measured slope of α=2.5+0.1 ppm/mM is similar with that previously reported (Rubin, S. M., Spence, M. M., Dimitrov, I. E., Ruiz, E. J., Pines, A. & Wemmer, D. E. (2001), Detection of a Conformational Change in Maltose Binding Protein by ¹²⁹Xe NMR Spectroscopy. J. Am. Chem. Soc., 123, 8616-8617).

The ¹²⁹Xe chemical shifts of 4 mM xenon in MBP solutions are also plotted in FIG. 21b. The slope of the titration at 4 mM xenon (α=2.4±0.1 ppm/mM) is similar to that at 1 mM xenon. The lack of xenon concentration dependence of α indicates that only a small fraction of the xenon interacts with any particular site in the protein (see below). The third titration plotted in FIG. 21b is for 10 mM xenon with denatured MBP; the slope (α=1.3±0.1 ppm/mM) is less than the slope of the native titrations. The α value for denatured MBP is less than that anticipated by previous studies of denatured proteins based on the number of amino acids in the polypeptide; however, deviations from the average value should be expected as contributions to the ¹²⁹Xe chemical shift from nonspecific interactions will depend on the amino acid composition of the chain (Rubin, S. M., Spence, M. M., Pines, A. & Wemmer, D. E. (2001), Characterization of the Effects of Nonspecific Xenon-Protein Interactions on ¹²⁹Xe Chemical Shifts in Aqueous Solution: Further Development of Xenon as a Biomolecular Probe. J. Magn. Reson., 152(1), 79-86).

### Crystal Structure of MBP Pressurized with Xenon

Considering nonspecific interactions alone, the accessibility of more residues to xenon interactions in denatured MBP should result in an α value that is greater than that measured for native MBP. However, as seen in FIG. 21, the measured α value of MBP is greater under native conditions. We previously suggested that this behavior in other proteins was caused by the presence of specific, higher affinity interactions that also contribute significantly to the ¹²⁹Xe chemical shift (Rubin, S. M., Spence, M. M., Pines, A. & Wemmer, D. E. (2001), Characterization of the Effects of Nonspecific Xenon-Protein Interactions on ¹²⁹Xe Chemical Shifts in Aqueous Solution: Further Development of Xenon as a Biomolecular Probe. J. Magn. Reson., 152(1), 79-86). In order to demonstrate the presence of a xenon binding site in MBP, we solved the crystal structure of the protein without maltose at 1.8 Å in the presence of xenon. As previously described, unliganded MBP crystallized in the space group P1 with one molecule in the unit cell (Sharff, A. J., Rodseth, L. E., Spurlino, J. C. & Quiocho, F. A. (1992), Crystallographic Evidence of a Large Ligand-Induced Hinge-Twist Motion Between the Two Domains of the Maltodexrin Binding Protein Involved in Active Transport and Chemotaxis. Biochemistry, 31, 10657-10663 and Shilton, B. H., Shuman, H. A. & Mowbray, S. L. (1996), Crystal Structures and Solution Conformations of a Dominant-Negative Mutant of Escherichia Coli Maltose-Binding Protein. J. Mol. Biol., 2, 364-376). Crystals pressurized with 8 atm of xenon for 15 min were isomorphous to those previously used to solve the structure of unliganded MBP (PDB code 1OMP) (Sharff, A. J., Rodseth, L. E., Spurlino, J. C. & Quiocho, F. A. (1992), Crystallographic Evidence of a Large Ligand-Induced Hinge-Twist Motion Between the Two Domains of the Maltodexrin Binding Protein Involved in Active Transport and Chemotaxis. Biochemistry, 31, 10657-10663). The data collection and refinement statistics are summarized in Table 1.

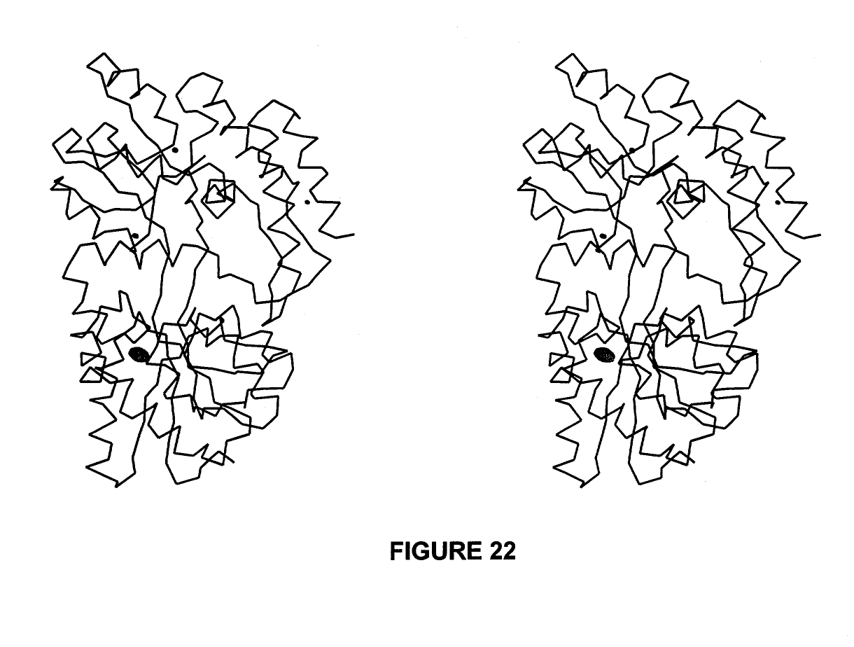

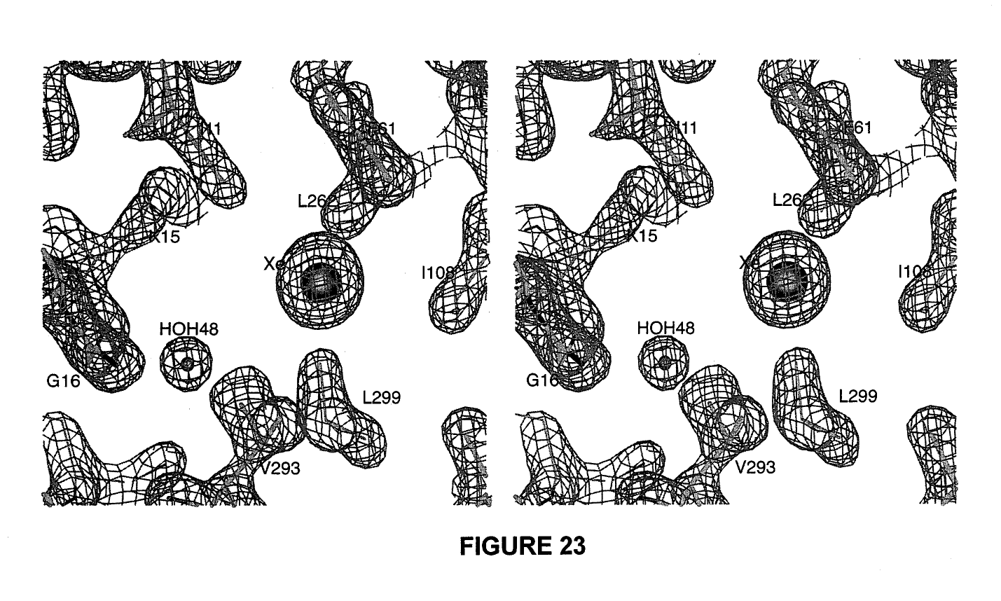

FIG. 22 shows a difference electron density map calculated with ||F_{obs, Xe}|−|F_{obs, native}|| using native structure factors and phases from the published structure (1OMP) (Sharff, A. J., Rodseth, L. E., Spurlino, J. C. & Quiocho, F. A. (1992), Crystallographic Evidence of a Large Ligand-Induced Hinge-Twist Motion Between the Two Domains of the Maltodexrin Binding Protein Involved in Active Transport and Chemotaxis. Biochemistry, 31, 106, 57-10663). Only one significant peak is present, with a height of 5σ, consistent with a single xenon atom. The occupancy of xenon in the final refined model is 0.5. The xenon binding site, shown in FIG. 23, is located in the N-terminal domain just below the surface of the sugar-binding cleft that is in contact with the reducing glycosyl unit in the maltose-bound structure. Table 2 lists the protein atoms that delimit the cavity and the distances between these atoms and xenon.

A water molecule and lysine side chain lining the cavity indicate the proximity of the cavity to the surface. This feature of the cavity is less common among xenon sites in proteins, most of which are more deeply buried (Prangé, T., Schiltz, M., Pernot, L., Colloc'h, N., Longhi, S., Bourget, W. & Fourme, R. (1998), Exploring Hydrophobic Sites in Proteins with Xenon and Krypton. Struct. Func. Gen., 30, 61-73). In the closed, maltose-bound state, the lysine forms two hydrogen bonds to the sugar (Spurlino, J. C., Lu, G. -Y. & Quiocho, F. A. (1991), The 2.3-Å Resolution Structure of the Maltose- or Maltodextrin-Binding Protein, a Primary Receptor of Bacterial Active Transport and Chemotaxis. J. Biol. Chem., 266(8), 5202-5219). The water molecule is relatively well ordered (refined B factor=18 Å²) and also appears in the open native structure (Sharff, A. J., Rodseth, L. E., Spurlino, J. C. & Quiocho, F. A. (1992), Crystallographic Evidence of a Large Ligand-Induced Hinge-Twist Motion-Between the Two Domains of the Maltodexrin Binding Protein Involved in Active Transport and Chemotaxis. Biochemistry, 31, 10657-10663). The bound xenon is localized in the cavity away from the lysine and water at the protein surface.

Comparison of the xenon bound structure with the native protein structure (1OMP) reveals little change in the position of the residues lining the binding cavity. The root mean squared deviation (RMSD) between the side chain atom positions of these residues (listed in Table 2) in the two structures is only 0.25 Å. For comparison, the RMSD between these same atoms in two MBP structures in the Protein Data Bank that have the same “open” conformation (1OMP and 1MPC) is 0.24 Å. The small perturbation in structure upon binding xenon is similar to what has been found in many proteins (Prangé, T., Schiltz, M., Pernot, L., Colloc'h, N., Longhi, S., Bourget, W. & Fourme, R. (1998), Exploring Hydrophobic Sites in Proteins with Xenon and Krypton. Struct. Func. Gen., 30, 61-73 and Stowell, M. H. B., Soltis, S. M., Kisker, C., Peters, J. W., Schindelin, H., Rees, D.C., Cascio, D., Beamer, L., Hart, P. J., Wiener, M. C. & Whitby, F. G. (1996), A Simple Device for Studying Macromolecular Crystals Under Moderate Gas Pressures (0.1-10 MPa). J. Appl. Crystallogr., 29, 608-613) and is consistent with the isomorphism of the crystals in the absence and presence of 8 atm xenon.

### Affinity of Xenon for MBP Changes with Protein Conformation

While the α values of MBP in the absence of ligand and bound to β-cyclodextrin are similar, the α value of the maltose-MBP complex is significantly less (Rubin, S. M., Spence, M. M., Dimitrov, I. E., Ruiz, E. J., Pines, A. & Wemmer, D. E. (2001), Detection of a Conformational Change in Maltose Binding Protein by ¹²⁹Xe NMR Spectroscopy. J. Am. Chem. Soc., 123, 8616-8617). Comparison of the x-ray structures of unliganded MBP and the maltose complex revealed a conformational change characterized as a rigid-body hinge bending between the two domains of the protein (Sharff, A. J., Rodseth, L. E., Spurlino, J. C. & Quiocho, F. A. (1992), Crystallographic Evidence of a Large Ligand-Induced Hinge-Twist Motion Between the Two Domains of the Maltodexrin Binding Protein Involved in Active Transport and Chemotaxis. Biochemistry, 31, 10657-10663 and Spurlino, J. C., Lu, G. -Y. & Quiocho, F. A. (1991), The 2.3-Å Resolution Structure of the Maltose- or Maltodextrin-Binding Protein, a Primary Receptor of Bacterial Active Transport and Chemotaxis. J. Biol. Chem., 266(8), 5202-5219). While binding of maltose induces the change from the “open” to “closed” structure, both x-ray and solution NMR data have demonstrated that the conformation in the β-cyclodextrin complex more closely resembles the “open” unliganded structure (Sharff, A. J., Rodseth, L. E. & Quiocho, F. A. (1993), Refined 1.8-Å Structure Reveals the Mode of Binding of P-Cyclodextrin to the Maltodextrin Binding Protein. Biochemistry, 32(40), 10553-10559 and Kay, L. E. (2001), Nuclear Magnetic Resonance Methods for High Molecular Weight Proteins: A Study Involving a Complex of Maltose Binding Protein and Beta-Cyclodextrin. Methods in Enzymology, 339(Part B), 174-203). This structural information with the ¹²⁹Xe NMR data suggests that the difference in α values results from the change in conformation. To test whether the affinity of xenon for the binding site in our crystal structure changes with conformation, we characterized xenon binding to MBP in the presence of β-cyclodextrin and maltose by heteronuclear NMR.

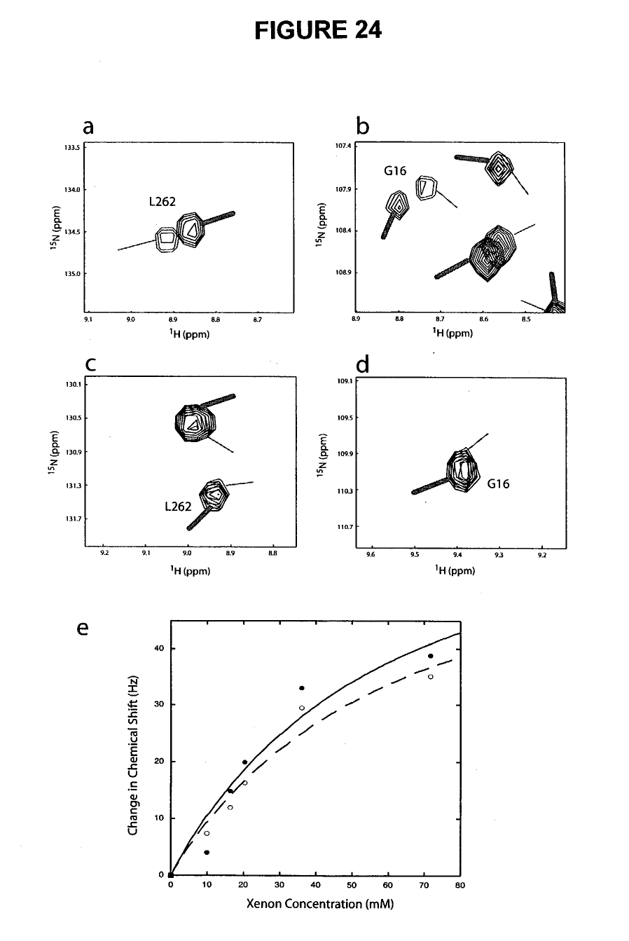

FIGS. 24a and 24b show portions of HSQC spectra of uniformly ¹⁵N labeled MBP in complex with β-cyclodextrin. The β-cyclodextrin complex was chosen instead of the unliganded protein because backbone resonance assignments have been reported (Gardner, K. H., Zhang, X., Gehring, K. & Kay, L. E. (1998), Solution NMR Studies of a 42 kDa Escherichia Coli Maltose Binding Protein/β-Cyclodextrin Complex: Chemical Shift Assignments and Analysis. J. Am. Chem. Soc., 120(45),11738-11748). Several peaks in the HSQC spectrum show small changes in ¹H and ¹⁵N chemical shift upon addition of xenon. With the available resonance assignments, the residues whose resonances shift can be mapped onto the structure. The peaks shown in FIGS. 24a and 24b correspond to Gly 16 and Leu 262, both of which line the xenon binding cavity seen in the crystal structure. The association constant of xenon can be estimated by measuring the changes in the HSQC spectrum as a function of xenon concentration in solution. The chemical shifts of both Gly 16 and Leu 262 are plotted in FIG. 24e; fits using a simple two site binding model yield association constants of 20±10 M⁻¹. This value is in rough agreement with the refined occupancy of xenon in the crystal structure at 8 atm of xenon, assuming a solubility of xenon in solution of 4.4 mM per atm overpressure.

A similar experiment was conducted with ¹⁵N labeled MBP in complex with maltose (FIGS. 24c and 24d). From reported assignments for this conformation (L. E. Kay (2002). Private Correspondence with Inventors),³⁸ the peaks corresponding to Gly16 and Leu22 were identified. These resonances show almost no change upon addition of xenon, indicating a lower affinity of xenon for the maltose complex at the binding site identified in the crystal structure. Small shifts in other peaks, corresponding to residues away from this binding site, were observed upon addition of xenon in both spectra. These shifts suggest other weak binding sites with occupancies too low to observe in the crystal structure. Considering that the small changes in peak position were of the same magnitude for both complexes, it is likely that the change in conformation does not affect the affinity at these other sites, so they do not contribute significantly to the change in ¹²⁹Xe shift.

¹²⁹Xe Chemical Shifts of Xenon in Solution with T4 Lysozyme Cavity Mutants

The observed ¹²⁹Xe chemical shift of xenon in protein solutions is determined by contributions from all specific and nonspecific xenon-protein interactions. The subject strategy for revealing the contribution of a particular interaction entails altering the accessibility of xenon to a single binding site while the rest of the interactions in solution remain constant. The crystal structures of wild type T4 lysozyme (WT*), and the variants L99A and L121A with xenon bound to internal cavities have been reported (Quillin, M. L., Breyer, W. A., Griswold, I. J. & Matthews, B. W. (2000), Size Versus Polarizability in Protein-Ligand Interactions: Binding of Noble Gases within Engineered Cavities in Phage T4 Lysozyme. J. Mol. Biol., 302, 955-977). Structures of L99A with benzene and n-butylbenzene bound in its cavity have also been determined (Morton, A. & Matthews, B. W. (1995), Specificity of Ligand Binding in a Buried Nonpolar Cavity of T4 Lysozyme: Linkage of Dynamics and Structural Plasticity. Biochemistry, 34(27), 8576-8588). Table 3 summarizes the volumes and xenon occupancies of these cavities in the structures (Quillin, M. L., Breyer, W. A., Griswold, I. J. & Matthews, B. W. (2000), Size Versus Polarizability in Protein-Ligand Interactions: Binding of Noble Gases within Engineered Cavities in Phage T4 Lysozyme. J. Mol. Biol., 302, 955-977).

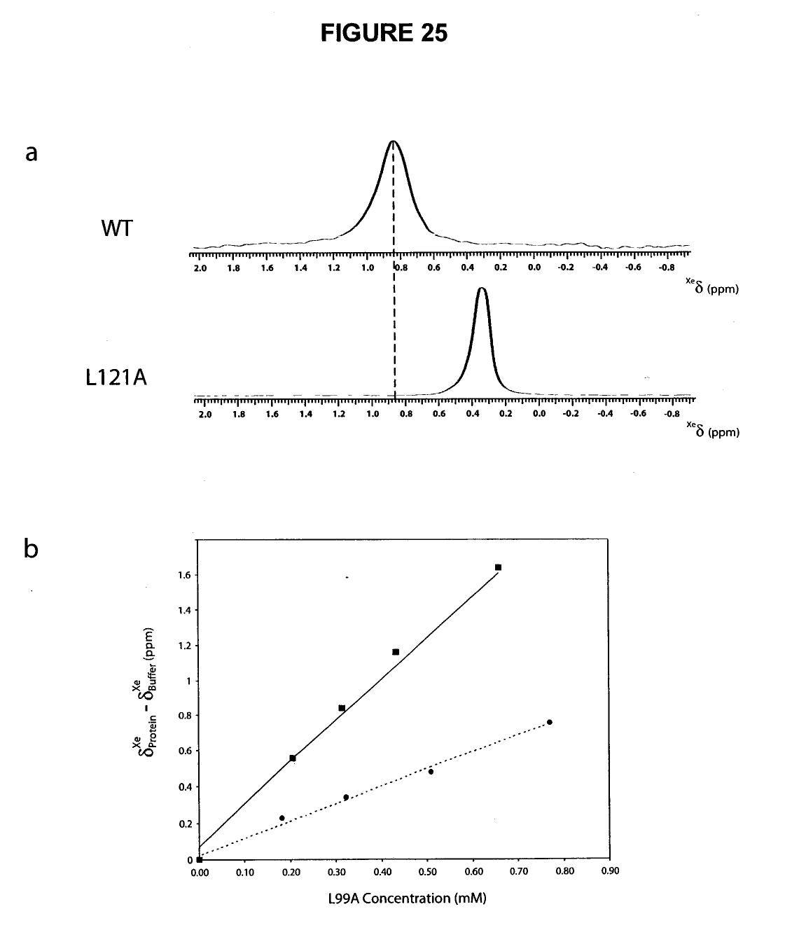

We measured ¹²⁹Xe chemical shifts of xenon in solutions of these proteins. FIG. 25a shows ¹²⁹Xe NMR spectra of 1.5 mM xenon in solution with WT* and L121A T4 lysozyme at a protein concentration of 0.3 mM; results of the protein titration are plotted in FIG. 25b. Because the proteins are identical except for the structure of the xenon binding cavity, the change in α value between WT* (α=2.3±0.9 ppm/mM) and L121A (α=0.9±0.1 ppm/mM) can be attributed to different contributions to the overall shift from this site.

The structure of L99A at 8 atm xenon revealed two primary binding sites (numbered 1 and 2 in order of decreasing xenon occupancy) and a third with much lower xenon occupancy (Quillin, M. L., Breyer, W. A., Griswold, I. J. & Matthews, B. W. (2000), Size Versus Polarizability in Protein-Ligand Interactions: Binding of Noble Gases within Engineered Cavities in Phage T4 Lysozyme. J. Mol. Biol., 302, 955-977). To simplify data analysis and discussion, we consider only the two primary sites with any effects of site 3 subsumed by site 1. This approximation should be good considering that the xenon affinity for site 3 is much lower and that contributions from all sites to the overall chemical shift are additive (see below). Structures of L99A in the presence of benzene and n-butylbenzene show that these organic ligands bind to the same interior cavity as xenon, with affinities measured by calorimetric analysis of 5.7×10³ M⁻¹ and 7.0×10⁴ M⁻¹ respectively (Quillin, M. L., Breyer, W. A., Griswold, I. J. & Matthews, B. W. (2000), Size Versus Polarizability in Protein-Ligand Interactions: Binding of Noble Gases within Engineered Cavities in Phage T4 Lysozyme. J. Mol. Biol., 302, 955-977 and Morton, A. & Matthews, B. W. (1995), Specificity of Ligand Binding in a Buried Nonpolar Cavity of T4 Lysozyme: Linkage of Dynamics and Structural Plasticity. Biochemistry, 34(27), 8576-8588). The structures indicate that binding of benzene should inhibit binding of xenon to site 1, and binding of n-butylbenzene should inhibit binding of xenon to both sites. Thus, comparison of ¹²⁹Xe chemical shifts for titrations with L99A in the absence and presence of these ligands can resolve the contributions to the observed shifts from each specific site.

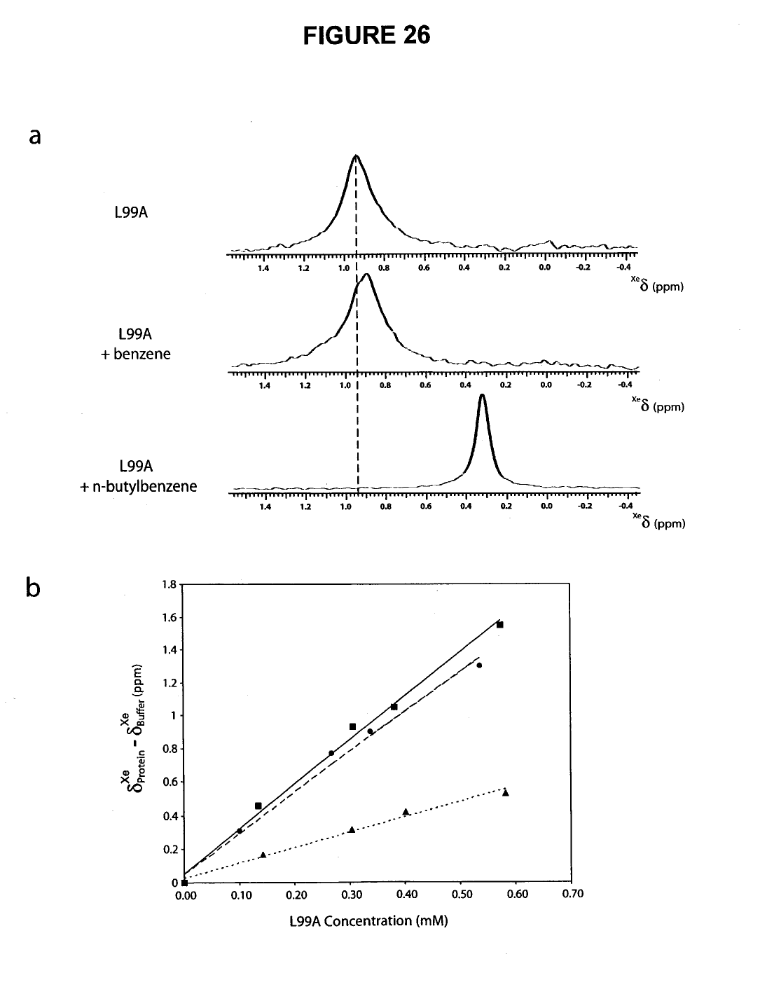

¹²⁹Xe NMR spectra of 1.5 mM xenon in solution with 0.3 mM L99A alone and with benzene and n-butylbenzene are shown in FIG. 26a. The addition of benzene has a small effect, while addition of n-butylbenzene has a larger effect on both the shift and width of the xenon resonance. The significant decrease in peak width with n-butylbenzene indicates that the spin interaction and/or exchange contribution to spin-spin relaxation have been reduced. Full titrations are shown in FIG. 26b for L99A. The modest change in slope between the titrations with (α=2.4±0.1 ppm/mM) and without (α=2.7±0.1 ppm/mM) benzene indicates that site 1 contributes only a small amount to the overall shift. The slope of the L99A+benzene titration (α=2.4±0.1 ppm/mM) is similar to the slope of the wild type titration (α=2.3±0.1 ppm/mM). The benzene insensitive site (site 2) is in the same cavity as the wild type site, hence binding induced shifts are expected to be similar. The α value for L99A is significantly less (α=0.9±0.1 ppm/mM) in the presence of n-butylbenzene, indicating a significant contribution to the observed shift from site 2.

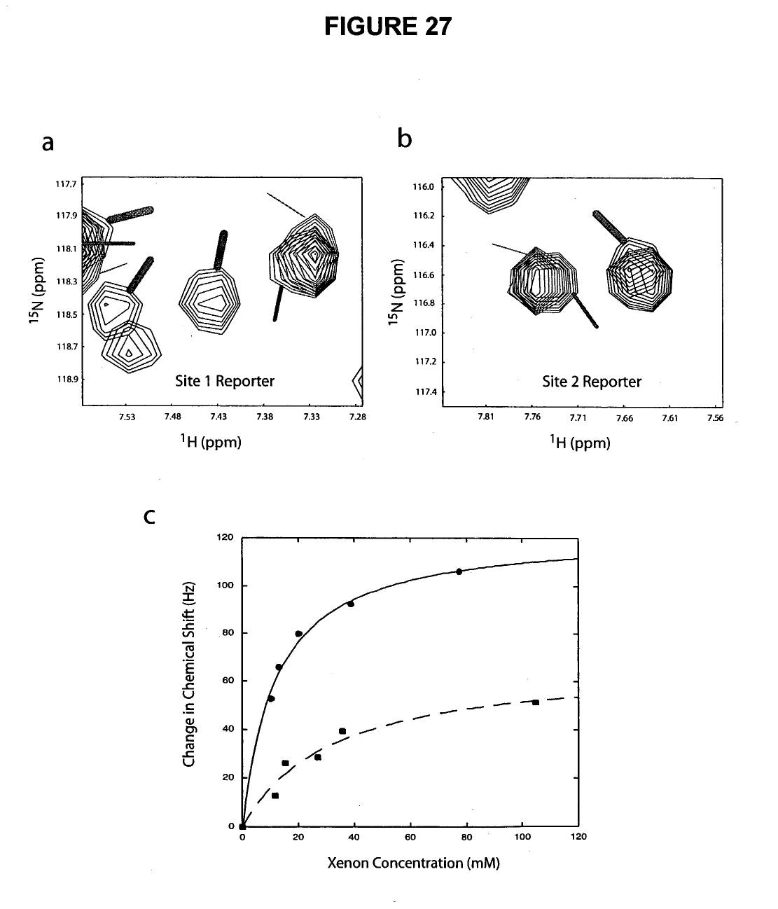

As discussed further below, determination of the absolute chemical shift of xenon bound to a cavity requires a separate measurement of the binding affinity. We have measured association constants of xenon with L99A in the absence and presence of benzene by monitoring changes of protein resonance chemical shifts with xenon concentration. Backbone and side chain dynamics of L99A in the presence of xenon have previously been studied by heteronuclear NMR; however, xenon affinities were not reported (Mulder, F. A. A., Hon, B., Muhandiram, D. J., Dahlquist, F. W. & Kay, L. E. (2000), Flexibility and Ligand Exchange in a Buried Cavity Mutant of T4 Lysozyme Studied by Multinuclear NMR. Biochemistry, 39(41), 12614-12622). FIG. 27 shows portions of the HSQC spectra of a sample containing 1.2 mM ¹⁵N labeled L99A with and without both xenon and benzene. Comparison of peak positions in the four spectra yields two patterns for changes in chemical shift upon addition of the ligands. Some resonances, exemplified by the peaks in FIG. 27a, show changes in chemical shift upon addition of benzene but have the same limiting shift upon titration of xenon. These peaks likely correspond to amide protons near site 1, which is the benzene binding site. The identical limiting shifts in the presence of xenon suggest that xenon can compete with benzene for the same binding site. However, comparison of affinity constants determined from these site 1 reporters in the absence and presence of benzene indicates that the affinity of xenon for site 1 is five times less with the amount of benzene present for ¹²⁹Xe NMR experiments. The titration curve without benzene for the site 1 reporter in FIG. 27a is shown in FIG. 27c. Peaks following the second pattern (FIG. 27b) show little or no change in chemical shift upon addition of benzene and have similar shifts in the presence and absence of benzene upon addition of saturating concentrations of xenon. In addition, the affinity of xenon deduced from these resonances does not change with addition of benzene. These peaks likely correspond to amide protons near site 2 in the protein, at some distance from the benzene binding site. The relevant affinity constant for site 2 reporters is that measured in the presence of benzene, because the effect of inhibiting site 2 on the ¹²⁹Xe chemical shift was observed with benzene present. The titration curve in the presence of benzene for the site 2 reporter in FIG. 27b is shown in FIG. 27c. A summary of the association constants measured from the two sets of reporters is given in Table 4. The reported values are the average of fits of chemical shift data from three reporters in each set; the reported error is the associated standard deviation for the three derived values.

### Detecting Xenon Binding Sites in Proteins by ¹²⁹Xe Chemical Shift Data

The changes in ¹²⁹Xe chemical shift with protein concentration can be described by a model that treats all specific and nonspecific interactions as weak binding sites. In general, the observed shift (δ_{obs}), referenced to the shift in buffer, can be written as the average chemical shift of xenon in each site (δᵢ) weighted by the occupancy,

$${δ}_{obs} = {\sum }_{i} {δ}_{i} \frac{{[ Xe ]}_{i}}{{[ Xe ]}_{total}} = {\sum }_{i} {δ}_{i} \frac{{K}_{i} ( {[ Xe ]}_{total} - {[ Xe ]}_{i} ) ( {[ Protein ]}_{total} - {[ Xe ]}_{i} )}{{[ Xe ]}_{total}} \qquad [ 1 ]$$

where δᵢ is also referenced to the shift in buffer and Kᵢ is the association constant of xenon with each site. In the limit that a small amount of xenon is bound to each site, (i.e. [Xe]ᵢ<<[Xe]ₜₒₜₐₗ, [Protein]ₜₒₜₐₗ) the above equation reduces to

$${δ}_{obs} = {\sum }_{i} {δ}_{i} {{K}_{i} [ Protein ]}_{total} = {α [ Protein ]}_{total} \\ where \\ α = {\sum }_{i} {δ}_{i} {K}_{i} \qquad [ 2 ]$$

Thus, the slopes of protein titration data such as those shown in FIGS. 21, 25, and 26 are indicative of the affinity and effect on the shift of all xenon-protein interactions in solution. Due to the small values of Kᵢ and the low total xenon and protein concentrations typically used, these titrations are indeed conducted in the limit in which a small fraction of xenon is bound. As seen in Eq. 2 and the data in FIG. 21, in this limit the observed slope a is independent of the xenon concentration in solution.

Considering that the contributions to α from nonspecific interactions scale roughly with protein accessible surface area (Rubin, S. M., Spence, M. M., Pines, A. & Wemmer, D. E. (2001), Characterization of the Effects of Nonspecific Xenon-Protein Interactions on ¹²⁹Xe Chemical Shifts in Aqueous Solution: Further Development of Xenon as a Biomolecular Probe. J. Magn. Reson., 152(1), 79-86), deviations from anticipated values of a suggest the presence of specific binding interactions. In particular, it appears that proteins with αₙₐₜᵢᵥₑ>α_{denatured} must contain a site that interacts more strongly with xenon than average surface sites because the surface area (and hence the number of nonspecific interactions) increases upon denaturation. The crystal structure of MBP with xenon reported here confirms the presence of the binding site predicted by comparison of the α values in FIG. 21. NMR and crystallographic data on other proteins confirm that proteins with αₙₐₜᵢᵥₑ>α_{denatured} bind xenon specifically; examples include the T4 lysozymes studied here, myoglobin (Rubin, S. M., Spence, M. M., Goodson, B. M., Wemmer, D. E. & Pines, A. (2000), Evidence of Nonspecific Surface Interactions Between Laser-Polarized Xenon and Myoglobin in Solution. Proc. Natl. Acad. Sci. USA, 97(17), 9472-9475 and Tilton, R. F., Jr., Kuntz, I. D., Jr. & Petsko, G. A. (1984), Cavities in Proteins: Structure of a Metmyoglobin-Xenon Complex Solved to 1.9 Å. Biochemistry, 23(13), 2849-2857), and bovine serum albumin (Wolber, J., Cherubini, A., Dzik-Jurasz, A. S., Leach, M. 0. & Bifone, A. (1999), Spin-lattice Relaxation of Laser-Polarized Xenon in Human Blood. Proc. Natl. Acad. Sci. USA, 96(7), 3664-9 and Rubin, S. M., Spence, M. M., Pines, A. & Wemmer, D. E. (2001), Characterization of the Effects of Nonspecific Xenon-Protein Interactions on ¹²⁹Xe Chemical Shifts in Aqueous Solution: Further Development of Xenon as a Biomolecular Probe. J. Magn. Reson., 152(1), 79-86). We note that in cases for which αₙₐₜᵢᵥₑ<α_{denatured} xenon binding cannot be precluded as the possibility exists for a specific site with a limiting shift that is upfield from the shift of xenon in buffer (i.e. δᵢ<0 in Eq. 2). Xenon binding in such cases may be confirmed by observing widths of ¹²⁹Xe resonances. Although nonspecific interactions result in some line broadening (Rubin, S. M., Spence, M. M., Pines, A. & Wemmer, D. E. (2001), Characterization of the Effects of Nonspecific Xenon-Protein Interactions on ¹²⁹Xe Chemical Shifts in Aqueous Solution: Further Development of Xenon as a Biomolecular Probe. J. Magn. Reson., 152(1), 79-86), the effects of specific interactions are more pronounced and can likely be distinguished.

¹²⁹Xe Shifts in Protein Cavities can be Determined in the Limit of Weak Binding and Correlate with Cavity Size

The sensitivity of the ¹²⁹Xe shift to its local environment has been exploited in studies of xenon interacting with materials such as zeolites, clathrates, and nanotubes (Ripmeester, J. A., Ratcliffe, C. I. & Tse, J. S. (1988), The Nuclear Magnetic Resonance of Xe-129 Trapped in Clathrates and Some Other Solids. Chem. Soc., Far. Trans., 84(11), 3731-3745, Bonardet, J., Fraissard, J., Gedeon, A. & Springuel-Huet, M. (1999), Nuclear Magnetic Resonance of Physisorbed 129-Xe Used as a Probe to Investigate Porous Solids. Cat. Rev.: Sci. Eng., 41(2), 115-225 and Sozzani, P., Comotti, A., Simonutti, R., Meersmann, T., Logan, J. W. & Pines, A. (2000), A Porous Crystalline Molecular Solid Explored by Hyperpolarized Xenon. Ang. Chem.-Int. Ed., 39(15), 2695-2698). Xenon can probe cavities in these structures, reporting on properties such as size, shape, chemical composition, and occupancy of the gas. Complementary information from crystallographic and ¹²⁹Xe NMR experiments has enabled interpretation of observations with hydrophobic cavities in proteins. The low affinity of xenon for cavities makes determination of the limiting shift of bound xenon (δₛᵢₜₑ) difficult. Fast exchange of xenon among all environments in solution prevents direct observation of δₛᵢₜₑ from a ¹²⁹Xe NMR spectrum. The limiting shift of xenon bound to myoglobin has been estimated from titrations in which a significant fraction of xenon is bound (Rubin, S. M., Spence, M. M., Goodson, B. M., Wemmer, D. E. & Pines, A. (2000), Evidence of Nonspecific Surface Interactions Between Laser-Polarized Xenon and Myoglobin in Solution. Proc. Natl. Acad. Sci. USA, 97(17), 9472-9475 and Locci, E., Dehouck, Y., Casu, M., Saba, G., Lai, A., Luhmer, M., Reisse, J. & Bartik, K. (2001), Probing Proteins in Solution by ¹²⁹Xe NMR Spectroscopy. J. Magn. Reson., 150(2), 167-174). However, this method is not ideal as it requires the addition of large amounts of protein to solution and the determination of shifts from broad spectral lines, nor is it general as xenon-protein interactions are typically too weak to attain substantial occupancy.

We demonstrate here that δₛᵢₜₑ can be determined even when a small fraction of xenon is bound by comparing titrations in which the interaction is available and blocked. As seen in Eq. 2, each contribution to the concentration normalized shift or α value of a protein depends on both δₛᵢₜₑ and the affinity (Kₛᵢₜₑ); thus, an independent measurement of the binding constant allows calculation of δₛᵢₜₑ. For example, the difference in α values between the L99A and L99A+benzene titrations marks the contribution from site 1 in the protein (i.e. from Eq. 2, Δα=δ_{site 1}K_{site 1}). Using the association constant (K_{site 1}=90±40 M⁻¹) obtained from the HSQC titration (FIG. 27), the ¹²⁹Xe chemical shift of xenon bound to the cavity relative to the shift in buffer (δ_{site 1}=3±4 ppm) and the absolute shift referenced to xenon gas in the limit of zero density (δ*=198±4 ppm) can be estimated. Table 4 gives the limiting shift derived for xenon bound to each cavity in L99A.

Comparison of the α values of wild type and cavity mutants of T4 lysozyme reveals that smaller cavities give rise to larger downfield ¹²⁹Xe shifts than those from larger cavities. Replacing Leu 121 in T4 lysozyme with alanine enlarges the xenon binding cavity from 58 Å³ to 165 Å³; this change in structure induces a change in α values from 2.3 to 0.9 ppm/mM as seen in FIG. 25. While contributions from nonspecific interactions remain the same, the contribution to α from this specific interaction at the cavity is greater in the wild type protein (i.e. δ_{WT*}K_{WT*}>δ_{L121A}K_{L121A}). The refined occupancy of xenon in each cavity is 0.7 for crystals of each protein at 8 bar of xenon (Quillin, M. L., Breyer, W. A., Griswold, I. J. & Matthews, B. W. (2000), Size Versus Polarizability in Protein-Ligand Interactions: Binding of Noble Gases within Engineered Cavities in Phage T4 Lysozyme. J. Mol. Biol., 302, 955-977). Assuming that the same crystallographic occupancies indicate similar affinities in solution (K_{WT*}˜K_{L121A}), the ¹²⁹Xe shift of xenon bound to the smaller cavity of the wild type protein must be further downfield than xenon bound to L121A (Δ_{WT*}>δ_{L121A}). Titration data for L99A indicate that xenon interactions with the smaller site 2 cavity (47 Å³) result in a larger downfield contribution to the a value than interactions with the larger site 1 cavity (115 Å³). The similar affinities determined from the HSQC data indicate that these different contributions are due to different values of the limiting shift of bound xenon (i.e. δₛᵢₜₑ₂=30±10 ppm>δₛᵢₜₑ₁=3±4 ppm). The observation that smaller cavities in proteins induce larger downfield ¹²⁹Xe shifts is consistent with studies of xenon in water clathrates and zeolites (Ripmeester, J. A., Ratcliffe, C. I. & Tse, J. S. (1988), The Nuclear Magnetic Resonance of Xe-129 Trapped in Clathrates and Some Other Solids. Chem. Soc., Far. Trans., 84(11), 3731-3745 and Bonardet, J., Fraissard, J., Gédéon, A. & Springuel-Huet, M. (1999), Nuclear Magnetic Resonance of Physisorbed 129-Xe Used as a Probe to Investigate Porous Solids. Cat. Rev.: Sci. Eng., 41(2), 115-225).

Change in Structure of the Xenon Binding Site Correlates with the Sensitivity of the ¹²⁹Xe Chemical Shift to MBP Conformation

Addition of maltose to native MBP induced an almost twofold change in the α value (Rubin, S. M., Spence, M. M., Dimitrov, I. E., Ruiz, E. J., Pines, A. & Wemmer, D. E. (2001), Detection of a Conformational Change in Maltose Binding Protein by ¹²⁹Xe NMR Spectroscopy. J. Am. Chem. Soc., 123, 8616-8617). The sensitivity of the ¹²⁹Xe shift to native state conformation suggests the possibility of using xenon to detect protein functional states or ligand binding events. Understanding the structural basis for the change in chemical shift with conformation is necessary to extend such an assay to other biomolecular systems. The twofold difference in α between the protein states is too large to result from a change in the chemical composition or accessibility of the protein surface. Rather, we proposed that the difference resulted from altered interactions at a specific xenon binding site (Rubin, S. M., Spence, M. M., Dimitrov, I. E., Ruiz, E. J., Pines, A. & Wemmer, D. E. (2001), Detection of a Conformational Change in Maltose Binding Protein by ¹²⁹Xe NMR Spectroscopy. J. Am Chem. Soc., 123, 8616-8617). The crystal structure of unliganded MBP with xenon confirms the presence of such a binding site. Because MBP induced ¹²⁹Xe shifts depend on the bound shift and the binding constant, a change in either parameter upon binding maltose would explain the change in α values. The xenon titration followed in the HSQC spectra (FIG. 24) indicates that the affinity of xenon for MBP is greater in the open conformation than the closed conformation; this difference in affinity is likely due to a change in the structure of the xenon binding cavity that occurs when MBP binds maltose, but not β-cyclodextrin.

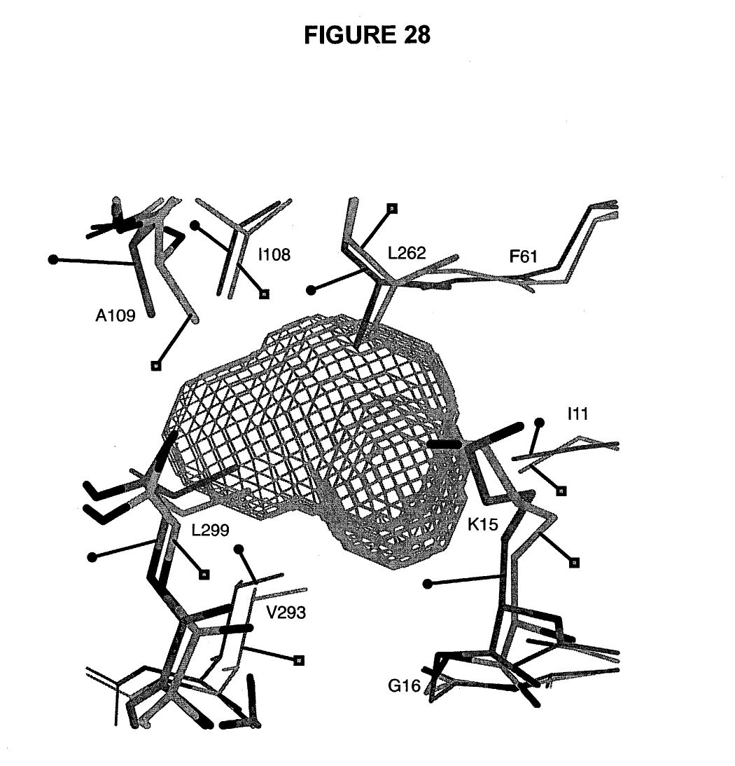

FIG. 28 shows the xenon binding cavity in the published structures of MBP complexed with β-cyclodextrin (PDB code: 1DMB) and maltose (PDB code: 1ANF) (Sharff, A. J., Rodseth, L. E. & Quiocho, F. A. (1993), Refined 1.8-Å Structure Reveals the Mode of Binding of β-Cyclodextrin to the Maltodextrin Binding Protein. Biochemistry, 32(40),10553-10559 and Quiocho, F. A., Spurlino, J. C. & Rodseth, L. E. (1997), Extensive Features of Tight Oligosaccharide Binding Revealed in High-Resolution Structures of the Maltodextrin Transport/Chemosensory Receptor. Structure, 5(8), 997-1015). The two structures were superimposed by minimizing C_{α}, distances between residues in the N-terminal domain (5-109, 284-319). The cavity space found by VOIDOO in the β-cyclodextrin structure is shown in the central cross-hatched region (Kleywegt, G. J. & Jones, T. A. (1994), Detection, Delineation, Measurement, and Display of Cavities in Macromoleclar Structures. Acta. Cryst., D50, 178-185). Comparison of the structures reveals only small changes in the positions of the residues that line the cavity. This similarity in cavity structure is not surprising considering that the cavity is located entirely within the N-terminal domain and that the overall conformation of the two complexes differs by a rigid body hinge bend of the two domains. The difference in the position of Lys 15 between the two structures, however, is prominent and results from the fact that the side-chain amino group hydrogen bonds specifically to maltose in the nearby sugar-binding cleft. This hydrogen bonding reduces the flexibility of the lysine, as seen in the lower refined B factors for the side-chain atoms in the maltose structure (˜10-20 Å²) relative to the unliganded and β-cyclodextrin structures (˜20-30 Å²). The change in lysine position results in a modest difference in calculated cavity volumes between the maltose bound structure (75 Å³) and the unliganded (95 Å³) and β-cyclodextrin (93 Å³) structures. It remains unclear whether this change in cavity volume is the cause of the lower affinity of xenon for the maltose complex, particularly considering that xenon localizes in the cavity away from the position of the lysine. However, the correlations between the ¹²⁹Xe chemical shift data, xenon-protein affinity, and the hydrogen bonding of the lysine strongly suggest that this residue plays a role in the mechanism for detection of the conformational change by ¹²⁹Xe NMR.

Materials and Methods

Protein Expression and Purification

Maltose binding protein from Escherichia Coli was expressed and purified as previously described (Rubin, S. M., Spence, M. M., Dimitrov, I. E., Ruiz, E. J., Pines, A. & Wemmer, D. E. (2001), Detection of a Conformational Change in Maltose Binding Protein by ¹²⁹Xe NMR Spectroscopy. J. Am. Chem. Soc., 123, 8616-8617). Wild type and mutant T4 lysozymes, all cysteine free variants, were expressed as described by Matthews and coworkers (Eriksson, A. E., Baase, W. A., Zhang, X. -J., Heinz, D. W., Blaber, M., Baldwin, E. P. & Matthews, B. W. (1992), Response of a Protein Structure to Cavity-Creating Mutations and Its Relation to the Hydrophobic Effect. Science, 255, 178-183), except L99A which was subcloned into a PET vector and expressed in the E. Coli strain BL21 (DE3). ¹⁵N labeled MBP and T4 lysozyme were isolated from E. Coli grown in M9 media with ¹⁵NH₄Cl as the only nitrogen source. All T4 lysozymes were purified using CM ion exchange resin (Pharmacia Biotech) in a 50 mM Tris buffer at pH 7.4. All purified proteins were dialyzed against water for three days, with a change of water twice a day prior to lyophilization.

¹²⁹Xe NMR Data Collection

Lyophilized protein samples were dissolved into 1 mL of buffer containing 100 mM Tris (pH 7.6 for MBP, pH 7.4 for the T4 lysozymes) and 20% D₂O. For the MBP titration under denaturing conditions, the buffer consisted of 100 mM citric acid (pH 3.0), 6M urea, and 20% D₂O. The protein stock solution was diluted with buffer to make four 400 μL samples at different concentrations; a fifth sample of 400 μL of buffer alone was used as a reference. Each sample in the L99A T4 lysozyme titrations done in the presence of organic molecules was prepared by addition of 0.5 μL of the neat organic ligand to the 400 μL of protein solution. Previous studies demonstrated that this preparation maintains sufficient ligand in solution to saturate the binding sites over the course of a short experiment such as ¹²⁹Xe NMR data collection (Morton, A., Baase, W. A., & Matthews, B. W. (1995), Energetic Origins of Specificity of Ligand Binding in an Interior Nonpolar Cavity of T4 Lysozyme. Biochemistry, 34, 8564-8575). Inconsistencies in the buffer composition of samples being compared can affect ¹²⁹Xe NMR chemical shifts. For this reason, many precautions were taken to ensure that the ionic strength, pH, and D₂O composition of the protein stock solution and buffer used for subsequent dilutions were kept the same. The use of KOH, HCl, and KCl to make any necessary adjustments is convenient because K⁺ and Cl⁻ have similar effects on the ¹²⁹Xe chemical shift (McKim, S. & Hinton, J. F. (1993), Xe-129 NMR Spectroscopic Investigation of the Interaction of Xenon with Ions in Aqueous Solution. J. Mag. Res. A, 104(3), 268-272).

A portion of the buffer (5-10 mL) was added to a glass bulb for mixing of laser-polarized xenon as previously described (Wolber, J., Cherubini, A., Dzik-Jurasz, A. S., Leach, M. 0. & Bifone, A. (1999), Spin-lattice Relaxation of Laser-Polarized Xenon in Human Blood. Proc. Natl. Acad. Sci. USA, 96(7), 3664-9). The bulb has an inlet that can be opened via a Teflon valve to a vacuum or the source of laser-polarized xenon and has a septum covered outlet, through which buffer can be withdrawn with a syringe. The buffer was degassed using three freeze-pump-thaw cycles prior to the introduction of laser-polarized gas in order to remove oxygen that could relax the xenon. Natural abundance ¹²⁹Xe (Isotech) was polarized with standard procedures. A pressure gauge was used to monitor the amount of gas transferred from the polarization apparatus to the bulb. The xenon concentration in the buffer is assumed to be linear with gas overpressure with a solubility of 4.4 mM/atm (Clever, H. L., Ed. (1979), Solubility Data Series. Vol. 2. New York: Pergamon). After vigorous shaking, 200 μL of buffer was withdrawn from the bulb and mixed rapidly with a protein sample in a standard 5 mM NMR tube. ¹²⁹Xe NMR spectra were acquired immediately following mixing for each of the five samples. Spectra were acquired at room temperature on a 300 MHz (proton frequency) Varian Inova spectrometer. The denatured MBP titration was done using unpolarized xenon as previously described for other denatured proteins (Rubin, S. M., Spence, M. M., Pines, A. & Wemmer, D. E. (2001), Characterization of the Effects of Nonspecific Xenon-Protein Interactions on ¹²⁹Xe Chemical Shifts in Aqueous Solution: Further Development of Xenon as a Biomolecular Probe. J. Magn. Reson., 152(1), 79-86). Following NMR data collection, protein sample concentrations were determined by absorbance at 280 nm (ε₂₈₀=66,350 M⁻¹ cm⁻¹ for MBP and ε₂₈₀=25,440 M⁻¹ cm⁻¹ for T4 lysozyme). Reported ¹²⁹Xe shifts are referenced to that of xenon in buffer alone; the chemical shift of xenon in buffer (except that containing 6M urea), referenced to the shift of xenon gas in the limit of zero density, was typically ≈195 ppm.

Xenon Affinity Determination

¹H-¹⁵N HSQC correlation spectra were acquired with uniformly ¹⁵N labeled MBP and T4 lysozyme L99A. MBP samples (0.4 mM) were prepared in buffer containing 20 mM sodium phosphate (pH 7.2), 100 μM EDTA, 10% D₂O and 2 mM of either β-cyclodextrin or maltose. T4 lysozyme samples (1.2 mM) were prepared in buffer containing 50 mM sodium phosphate (pH 5.8), 25 mM NaCl, 10% D₂O and 0.5 μL benzene where indicated. The presence of benzene was confirmed during the titration by observing its effect on the protein spectrum in the absence of xenon. Samples were in medium wall NMR tubes adapted with a screw-cap to allow xenon over pressures of several atmospheres. Samples were frozen and degassed once prior to addition of xenon, and pressures were measured with a gauge upon recovery of the gas after data acquisition. NMR spectra were acquired with 600 MHz and 500 MHz Bruker DRX spectrometers and processed using the NMRPipe and NMRView software packages (Johnson, B. A. & Blevins, R. A. (1994), NMRView: A Computer Program for the Visualization and Analysis of NMR Data. J. Biomol. NMR, 4, 603-614 and Delaglio, F., Grzesiek, S., Vuister, G., Zhu, G., Pfeifer, J. & Bax, A. (1995), NMRPipe: A Multidimensional Spectral Processing System Based on UNIX Pipes. J. Biomol. NMR, 6, 277-293). Titration data were fit to a two-site binding model using Kaleidagraph; reported errors in association constants correspond to either fitting errors or, in cases where chemical shift changes from a number of different resonances were averaged, standard deviations of a distribution.

Cavity Volume Calculations

Cavity volumes were calculated using VOIDOO (Kleywegt, G. J. & Jones, T. A. (1994), Detection, Delineation, Measurement, and Display of Cavities in Macromoleclar Structures. Acta. Cryst., D50, 178-185). We found that calculated volumes were often sensitive to the parameter values chosen for the grid size and probe radius parameters. In general, cavity searches with VOIDOO yielded no cavities when the van der Waals radius of xenon (2.2 Å) was used for the probe size. For calculations of cavities in T4 lysozyme L99A, grid (0.5 Å) and probe (radius=1.4 Å) parameters were chosen such that a sample calculation of the volume of the WT* cavity was consistent with that previously reported (Quillin, M. L., Breyer, W. A., Griswold, I. J. & Matthews, B. W. (2000), Size Versus Polarizability in Protein-Ligand Interactions: Binding of Noble Gases within Engineered Cavities in Phage T4 Lysozyme. J. Mol. Biol., 302, 955-977). Calculations of the cavity volumes of site 1 and site 2 in L99A were done by removing the xenon atom within the cavity under consideration from the PDB coordinate file. For consistency, cavities in MBP were calculated using similar parameters, except a probe radius of 1.45 Å was needed to close the cavity from the grid beyond the protein surface.

Crystal Structure Determination

Crystallization of unliganded MBP was done under similar conditions to those previously reported (Shilton, B. H., Shuman, H. A. & Mowbray, S. L. (1996), Crystal Structures and Solution Conformations of a Dominant-Negative Mutant of Escherichia Coli Maltose-Binding Protein. J. Mol. Biol., 2, 364-376). Crystals were obtained at room temperature using the hanging-drop vapor-diffusion method. 2 μL protein solution (16 mg/ml) was mixed with an equal volume of well solution containing 10 mM sodium citrate and 23% PEG 8000 (pH 6.6). Crystals with a size of 0.2×0.05×0.05 mm appeared in two days. Well solution with 10% glycerol was used as a cryoprotectant. Partial data sets collected for these crystals in the absence of xenon showed they were isomorphous with crystals used to solve previously reported structures (data not shown) (Sharff, A. J., Rodseth, L. E., Spurlino, J. C. & Quiocho, F. A. (1992), Crystallographic Evidence of a Large Ligand-Induced Hinge-Twist Motion Between the Two Domains of the Maltodexrin Binding Protein Involved in Active Transport and Chemotaxis. Biochemistry, 31, 10657-10663 and Shilton, B. H., Shuman, H. A. & Mowbray, S. L. (1996), Crystal Structures and Solution Conformations of a Dominant-Negative Mutant of Escherichia Coli Maltose-Binding Protein. J. Mol. Biol., 2, 364-376). Crystals were pressurized with xenon gas at 8 atm for 15 minutes and 12 atm for 30 minutes using a commercially available chamber (Hampton Research) prior to freezing in liquid nitrogen. Pressures were chosen based on the calculated affinity in solution from the NMR data. For an exposure time of 15 min at 8 atm, crystal diffracted in space group P1 with dimensions a=38.2 Å, b=44.0 Å, c=57.6 Å, α=100.7°, β=101.4°, γ=102.6°. For an exposure time of 30 min at 12 atm, crystals lost isomorphism (space group P1, cell dimensions a=52.2 Å, b=58.3 Å, c=64.9 Å, α=89.3°, β=82.40, γ=71.80) and had higher mosaicity. The adverse effects of the longer exposure time have been reported elsewhere and are likely a result of crystal drying despite saturation of the chamber with vapor from the mother liquor (Whittington, D. A., Rosenzweig, A. C., Frederick, C. A. & Lippard, S. J. (2001), Xenon and Halogenated Alkanes Track Putative Substrate Binding Cavities in the Soluble Methane Monooxygenase Hydroxylase. Biochemistry, 40, 3476-3482).

The diffraction data were collected at the Advanced Light Source (Berkeley, Calif.) using the 5.0.3 and 8.3.1 beam lines using an ADSC detector and a wavelength of 1.0 Å. A crystal-detector distance of 220 mm was used to collect data to a diffraction of 1.8 A (92.3% completion). The isomorphous data set (xenon exposure time 15 minutes at 8 atm) was integrated and scaled to 1.8 Å (92.3% completion) with MOSFLM (Delaglio, F., Grzesiek, S., Vuister, G., Zhu, G., Pfeifer, J. & Bax, A. (1995), NMRPipe: A Multidimensional Spectral Processing System Based on UNIX Pipes. J. Biomol. NMR, 6, 277-293) and the CCP4 suite (Collaborative Computational Project, N. (1994), The CCP4 Suite: Programs for Protein Crystallography. Acta Cryst. D, 55, 49-81) using the Elves interface (Holton, J. M. (2002), Elves: A User-Friendly Expert System for Macromolecular X-ray Crystallography. Manuscript in preparation). Unambiguous identification of electron density corresponding to xenon was made by generating a difference-Fourier map using structure factors from the unliganded structure in the Protein Data Bank (PDB code 1OMP). The map was generated by treating 1OMP as a native data set and our diffraction data set as a derivative. Structure refinement was done using CNS 1.0 (Brunger, A. T., Adams, P. D., Clore, G. M., DeLano, W. L., Gros, P., Grosse-Kunstleve, R. W., Jiang, J. S., Kuszewski, J., Nilges, M., Pannu, N. S., Read, R. J., Rice, L. M., Simonson, T. & Warren, G. L. (1998), Crystallography & NMR System: A New Software Suite for Macromolecular Structure Determination. Acta Cryst. D, 54, 905-921), beginning with 1 OMP as an initial model. Initial refinement consisted of rounds of rigid-body, simulated annealing, and B-group refinement. After the R factor and R_{free} reached 25% and 27% respectively, water and xenon were added to the model, followed by subsequent rounds of energy minimization, B-individual, and manual refinement. After the R factor and R_{free} reached 21% and 23%, occupancy of xenon was determined using a combination of occupancy and B-individual refinement. The process of occupancy refinement was repeated with a number of different initial xenon B factors; the final refined occupancy always fell within the range of 0.45-0.54. Data collection and refinement statistics are listed in Table 1. Manual refinement and graphics visualization were done with the 0 software package (Jones, T. A., Zou, J. -Y., Cowan, S. W. & Kjeldgaard, M. (1991), Improved Methods for Building Protein Models in Electron Density Maps and the Location of Errors in These Models. Acta Cryst. A, 47, 110-119). Geometry parameters were monitored using PROCHECK (Laskowski, R. A., MacArthur, M. W., Moss, D. S. & Thorton, J. M. (1993), PROCHECK—A Program to Check the Stereochemical Quality of Protein Structures. J. Appl. Crystallogr., 26, 283-291).

Accession Code

Coordinates and structure factors for the unliganded MBP crystal structure with xenon have been deposited in the Protein Data Bank under accession code 1LLS.

The invention has now been explained with reference to specific embodiments. Other embodiments will be suggested to those of ordinary skill in the appropriate art upon review of the present specification.

### References

It is noted that all of the references cited above are incorporated herein by reference. Once again, in particular, S. M. Rubin, M. M. Spence, B. M. Goodson, D. E. Wemmer, A. Pines, Proceedings of the National Academy of Sciences of the United States of America 97, 9472-9475 (2000) and Rubin, S. M., Spence, M. M. Pines, A. and Wemmer, D. E. (2001) J. Magn. Reson., 152(1), 79-86 were in the original subject Provisional Application No. 60/399,041 and are specifically incorporated herein by reference, as are all of the Provisional Application file documents.

Although the description above contains many specificities, these should not be construed as limiting the scope of the invention but as merely providing illustrations of some of the presently preferred embodiments of this invention. Therefore, it will be appreciated that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. 112, sixth paragraph, unless the element is expressly recited using the phrase “means for.”

TABLE 1
Crystallographic Data Collection and Refinement Statistics

| Crystal: | |
|---|---|
| Space Group | P1 |
| Cell dimensions a, b, c (Å) | 38.2 44.0 57.6 |
| Cell dimensions α, β, γ (deg) | 100.7 101.4 102.6 |
| Data collection (cryogenic): | |
| Resolution Limit (Å) | 17.07-1.80 |
| Measured Reflections | 55, 908 |
| Unique Reflections | 29, 855 |
| R_{sym} (%) | 2.9 |
| Completeness (%) | 92.3 |
| Ave. B-factor (Å²) (Wilson 1.8-3.0 Å) | 16.9 |
| Refinement: | |
| No. of Molecules in AU | 1 |
| No. of Amino Acids | 370 |
| No. of Solvent | 172 |
| Resolution Used | 17.07-1.80 |
| Sigma Cutoff | 0.0 |
| No. of Reflections Work/Test | 28,283/1472 |
| Final R factor/R_{free} (\|F\| > 0σ)(%) | 20.4/22.6 |
| Average B Factor (Å²) | 22.5 |
| Highest Resolution Bin | 1.80-1.91 |
| Completeness Highest Resolution Bin | 76% |
| R factor/R_{free} Highest Resolution Bin | 24.1/25.8 |
| R.M.S. Deviations from Ideal Geometry: | |
| Bond Length (Å) | 0.010 |
| Bond Angles (deg) | 1.4 |

TABLE 2
Distances from Xenon to Neighboring Atoms in Maltose Binding
Protein

| Residue | Atom | Distance to Xe (Å) |
|---|---|---|
| ILE 11 | Cγ₁ | 5.3 |
| ILE 11 | Cδ₁ | 3.8 |
| LYS 15 | Cβ | 5.9 |
| LYS 15 | Cγ | 6.4 |
| GLY 16 | O | 6.0 |
| LEU 20 | Cα | 6.0 |
| LEU 2O | Cβ | 5.1 |
| LEU 20 | Cγ | 5.0 |
| LEU 20 | Cδ₁ | 4.3 |
| LEU 20 | Cδ₂ | 4.7 |
| PHE 61 | Cβ | 5.5 |
| PHE 61 | Cγ | 4.7 |
| PHE 61 | Cδ₁ | 5.3 |
| PHE 61 | Cδ₂ | 3.8 |
| PHE 61 | Cε₁ | 5.1 |
| PHE 61 | Cε₂ | 3.6 |
| PHE 61 | Cξ | 4.3 |
| ILE 108 | Cβ | 5.1 |
| ILE 108 | Cγ₁ | 5.1 |
| ILE 108 | Cγ₂ | 4.1 |
| ILE 108 | Cδ₁ | 5.5 |
| LEU 262 | Cγ | 5.5 |
| LEU 262 | Cδ₁ | 6.3 |
| LEU 262 | Cδ₂ | 4.6 |
| LEU 284 | Cγ | 5.0 |
| LEU 284 | Cδ₁ | 4.5 |
| LEU 284 | Cδ₂ | 4.3 |
| LEU 290 | Cδ₂ | 5.7 |
| VAL 293 | Cα | 4.6 |
| VAL 293 | Cγ₁ | 4.0 |
| VAL 293 | Cγ₂ | 4.2 |
| LEU 299 | Cγ | 5.3 |
| LEU 299 | Cδ₁ | 5.2 |
| LEU 299 | Cδ₂ | 4.4 |
| HOH 48 | O | 5.6 |

TABLE 3
Xenon Binding Sites in T4 Lysozyme Cavity Mutants

| | | | Cavity Volume | Xenon |
|---|---|---|---|---|
| Protein | PDB Code | Site | (Å³) | Occupancy |
| Wild Type | 1C6T | 1 | 58^{a} | 0.6 |
| L121A | 1C65 | 1 | 165^{a} | 0.7 |
| L99A | 1C6K | 1 | 115^{b} | 0.7 |
| L99A | 1C6K | 2 | 41^{b} | 0.6 |

a) Previously reported (Quillin, M. L., Breyer, W. A., Griswold, I. J. & Matthews, B. W. (2000), Size Versus Polarizability in Protein-Ligand Interactions: Binding of Noble Gases within Engineered Cavities in Phage T4 Lysozyme. J. Mol. Biol., 302, 955-977)

b) Calculated as described in Materials and Methods

TABLE 4
Determination of ¹²⁹Xe Chemical Shift of Xenon Bound to T4
Lysozyme L99A

| | Titra- | | | | |
|---|---|---|---|---|---|
| | tion | | | | |
| Cav- | Com- | Δα = δₛᵢₜₑKₛᵢₜₑ | | | |
| ity | parison | (ppm/mM) | Kₛᵢₜₑ (M⁻¹)^{a} | Δδₛᵢₜₑ (ppm)^{b} | δ* (ppm)^{c} |
| Site | L99A | 0.3 ± 0.2 | 90 ± 40 | 3 ± 4 | 198 ± 4 |
| 1 | L99A + | | | | |
| | benzene | | | | |
| Site | L99A + | 1.5 ± 0.2 | 50 ± 10 | 30 ± 10 | 225 ± 10 |
| 2 | benzene | | | | |
| | L99A + | | | | |
| | n-butyl- | | | | |
| | benzene | | | | |

a) The reported affinity constant is the average constant determined from L99A HSQC data. For site 2, the affinity was determined in the presence of benzene.

b) Chemical shift relative to the shift of xenon in buffer. Error is estimated from the relative errors in the measurements of Δα and Kₛᵢₜₑ.

c) Absolute chemical shift referenced to the shift of xenon gas in the limit of zero density.

What is claimed is:

1. A method for detecting a conformational change or binding event in a targeted molecule, comprising:
(a) producing a magnetically active-nucleus sensor capable of producing a detectable signal when the targeted molecule undergoes a conformational change or binding event;
(b) combining said magnetically active-nucleus sensor with said targeted molecule; and
(c) recording said detectable signal upon said conformational change or binding event, wherein said sensor does not participate in the conformational change or binding event.

2. A conformational change detection method according to claim 1, wherein said magnetically active-nucleus sensor generates an NMR and/or MRI detectable signal upon a conformational change or binding event in the targeted molecule.

3. A conformational change detection method according to claim 1, wherein said magnetically active-nucleus sensor comprises either a non-functionalized active-nucleus sensor or a functionalized active-nucleus sensor complex that signals the conformational change or binding event in the targeted molecule.

4. A conformational change detection method according to claim 1, wherein magnetically active-nucleus sensor comprises a magnetically active gas.

5. A conformational change detection method according to claim 4, wherein said magnetically active gas is selected from a group consisting essentially of hyperpolarized xenon, sulfur hexafluoride, and hyperpolarized helium.

6. A conformational change detection method according to claim 2, wherein said detectable signal is selected from a group consisting essentially of chemical shifts and relaxation times.

7. A method for detecting a conformational change in a targeted macromolecule, comprising:
(a) producing a magnetically active-nucleus sensor capable of producing a detectable signal when the targeted macromolecule undergoes a conformational change;
(b) combining said magnetically active-nucleus sensor with said targeted macromolecule; and
(c) recording said detectable signal upon said conformational change, wherein said sensor does not participate in the conformational change.

8. A conformational change detection method according to claim 7, wherein said magnetically active-nucleus sensor generates an NMR and/or MRI detectable signal upon a conformational change in the targeted macromolecule.

9. A conformational change detection method according to claim 7, wherein said magnetically active-nucleus sensor comprises either a non-functionalized active-nucleus sensor or a functionalized active-nucleus sensor complex that signals the binding induced conformational change in the targeted macromolecule.

10. A conformational change detection method according to claim 7, wherein magnetically active-nucleus sensor comprises a magnetically active gas.

11. A conformational change detection method according to claim 10, wherein said magnetically active gas is selected from a group consisting essentially of hyperpolarized xenon, sulfur hexafluoride, and hyperpolarized helium.

12. A conformational change detection method according to claim 8, wherein said detectable signal is selected from a group consisting essentially of chemical shifts and relaxation times.

13. A method for detecting binding of a ligand to a targeted macromolecule, wherein the ligand binding produces a detectable conformational change in the targeted macromolecule or a detectable binding event, comprising:
(a) producing a magnetically active-nucleus sensor capable of producing a detectable signal when the targeted macromolecule undergoes a ligand-induced conformational change or upon the binding event;
(b) combining said magnetically active-nucleus sensor with said targeted macromolecule; and
(c) recording said detectable signal upon said ligand-induced conformational change or binding event, wherein said sensor does not participate in the conformational change or binding event.

14. A ligand binding detection method according to claim 13, wherein said magnetically active-nucleus sensor generates an NMR and/or MRI detectable signal upon a conformational change in the targeted macromolecule or binding event.

15. A ligand binding detection method according to claim 13, wherein said magnetically active-nucleus sensor comprises either a non-functionalized active-nucleus sensor or a functionalized active-nucleus sensor complex that signals the binding induced conformational change in the targeted macromolecule or binding event.

16. A ligand binding detection method according to claim 13, wherein magnetically active-nucleus sensor comprises a magnetically active gas.

17. A ligand binding detection method according to claim 16, wherein said magnetically active gas is selected from a group consisting essentially of hyperpolarized xenon, sulfur hexafluoride, and hyperpolarized helium.

18. A ligand binding detection method according to claim 14, wherein said detectable signal is selected from a group consisting essentially of chemical shifts and relaxation times.

19. A method for detecting a conformational change induced by a binding event in a targeted macromolecule, comprising:
(a) producing a magnetically active-nucleus sensor capable of producing a detectable signal when the targeted macromolecule undergoes the binding event induced conformational change;
(b) combining said magnetically active-nucleus sensor with said targeted macromolecule; and
(c) recording said detectable signal upon said binding event induced conformational change, wherein said sensor does not participate in the conformational change.

20. A binding event induced conformational change detection method according to claim 19, wherein said magnetically active-nucleus sensor generates an NMR and/or MRI detectable signal upon the binding event induced conformational change in the targeted macromolecule.

21. A binding event induced conformational change detection method according to claim 19, wherein said magnetically active-nucleus sensor comprises either a non-functionalized active-nucleus sensor or a functionalized active-nucleus sensor complex that signals the binding event induced conformational change in the targeted macromolecule.

22. A binding event induced conformational change detection method according to claim 19, wherein magnetically active-nucleus sensor comprises a magnetically active gas.

23. A binding event induced conformational change detection method according to claim 22, wherein said magnetically active gas is selected from a group consisting essentially of hyperpolarized xenon, sulfur hexafluoride, and hyperpolarized helium.

24. A binding event induced conformational change detection method according to claim 20, wherein said detectable signal is selected from a group consisting essentially of chemical shifts and relaxation times.

25. A method for detecting a binding event or environmental alteration induced conformational change in a targeted macromolecule, comprising:
(a) producing a magnetically active-nucleus sensor capable of producing a detectable signal when the targeted macromolecule undergoes the conformational change;
(b) combining said magnetically active-nucleus sensor with said targeted macromolecule; and
(c) recording said detectable signal upon said conformational change, wherein said sensor does not participate in the conformational change or binding event.

26. A conformational change detection method according to claim 25, wherein said magnetically active-nucleus sensor generates an NMR and/or MRI detectable signal upon the conformational change in the targeted macromolecule.

27. A conformational change detection method according to claim 25, wherein said magnetically active-nucleus sensor comprises either a non-functionalized active-nucleus sensor or a functionalized active-nucleus sensor complex that signals the conformational change in the targeted macromolecule.

28. A conformational change detection method according to claim 25, wherein magnetically active-nucleus sensor comprises a magnetically active gas.

29. A conformational change detection method according to claim 28, wherein said magnetically active gas is selected from a group consisting essentially of hyperpolarized xenon, sulfur hexafluoride, and hyperpolarized helium.

30. A conformational change detection method according to claim 26, wherein said detectable signal is selected from a group consisting essentially of chemical shifts and relaxation times.

31. A method for detecting a conformational change or a binding event in a targeted protein, comprising:
(a) producing a hyperpolarized ¹²⁹Xe sensor;
(b) combining said hyperpolarized ¹²⁹Xe sensor with said targeted protein; and
(c) recording from said hyperpolarized ¹²⁹Xe sensor an NMR and/or MRI detectable signal upon said conformational change or binding event, wherein said ¹²⁹Xe sensor does not participate in the conformational change or binding event.

32. A conformational change detection method according to claim 31, wherein said hyperpolarized ¹²⁹Xe sensor comprises either a non-functionalized ¹²⁹Xe sensor or a functionalized ¹²⁹Xe sensor complex that signals the conformational change in the targeted protein or binding event.

33. A conformational change detection method according to claim 31, wherein said detectable signal is selected from a group consisting essentially of chemical shifts and relaxation times.

34. A method for detecting a binding event or environmental alteration induced conformational change or binding event in a targeted protein, comprising:
(a) producing a hyperpolarized ¹²⁹Xe sensor;
(b) combining said hyperpolarized ¹²⁹Xe sensor with said targeted protein; and
(c) recording from said hyperpolarized ¹²⁹Xe sensor an NMR and/or MRI detectable signal upon said conformational change or binding event, wherein said ¹²⁹Xe sensor does not participate in the conformational change or binding event.

35. A conformational change detection method according to claim 34, wherein said hyperpolarized ¹²⁹Xe sensor comprises either a non-functionalized ¹²⁹Xe sensor or a functionalized ¹²⁹Xe sensor complex that signals the conformational change in the targeted protein or binding event.

36. A conformational change detection method according to claim 34, wherein said detectable signal is selected from a group consisting essentially of chemical shifts and relaxation times.

37. A method for detecting a conformational change or binding event in a targeted macromolecule, comprising:
(a) functionalizing a magnetically active nucleus by incorporating said nucleus into a macromolecular or molecular complex that is capable of binding the targeted macromolecule;
(b) bringing said macromolecular or molecular complex into contact with the targeted macromolecular; and
(c) detecting the occurrence, deletion, or change in a nuclear magnetic resonance signal from said functionalized nucleus in order to detect any conformational change in the targeted macromolecule or binding event, wherein said complex does not participate in the conformational change or binding event.

38. The method according to claim 37, wherein said binding to said target macromolecule is either in vivo or in vitro.

39. The method according to claim 37, wherein said macromolecule or molecular complex includes a structure selected from a group consisting essentially of monoclonal antibodies, other xenon binding proteins, dendrimers, self-assembled lipid complexes, liposomes, cyclodextrins, cryptands, cryptophanes, carcerands, microbubbles, micelles, vesicles, fullerenes, and molecular cage structures.

40. The method according to claim 37, wherein said macromolecular molecular complex includes a magnetically active gas contained within a molecular carrier.

41. The method according to claim 40, wherein said magnetically active gas is selected from a group consisting essentially of hyperpolarized xenon, sulfur hexafluoride, and hyperpolarized helium.

42. The method according to claim 37, wherein said magnetic resonance signal is selected from a group consisting essentially of chemical shifts and relaxation times.

43. A method for detecting conformational changes in a plurality of targeted macromolecules or binding events utilizing a plurality of functionalized active-nucleus sensor complexes with at least two of the functionalized active-nucleus sensor complexes having an attraction affinity to different corresponding targeted macromolecules, comprising:
(a) for each functionalized active-nucleus complex, functionalizing an active-nucleus by incorporating said active-nucleus into a macromolecular or molecular sensor complex that is capable of binding one of said targeted macromolecules;
(b) bringing said macromolecular or molecular sensor complexes into contact with the targeted macromolecules; and
(c) detecting the occurrence of or change in a nuclear magnetic resonance signal from each of said active-nuclei in each of said functionalized active-nucleus sensor complexes in order to detect conformational changes in the targeted macromolecules or binding event, wherein said complexes do not participate in the conformational change or binding event.

44. The method according to claim 43, wherein said binding to said target species is either in vivo or in vitro.

45. The method according to claim 43, wherein said functionalized active-nucleus complexes include structures selected from a group consisting essentially of monoclonal antibodies, other xenon binding proteins, dendrimers, self-assembled lipid complexes, liposomes, cyclodextrins, cryptands, cryptophanes, carcerands, microbubbles, micelles, vesicles, fullerenes, and molecular cage structures.

46. The method according to claim 43, wherein each said functionalized active-nucleus complex includes a magnetically active gas contained within a molecular carrier.

47. The method according to claim 46, wherein said magnetically active gas is selected from a group consisting essentially of hyperpolarized xenon, sulfur hexafluoride, and hyperpolarized helium.

48. The method according to claim 43, wherein said detecting comprises detecting the occurrence, deletion, or change in a magnetic resonance signal with a unique magnetic resonance property from each said functionalized active-nucleus sensor complex.

49. The method according to claim 48, wherein said magnetic resonance property is selected from a group consisting essentially of chemical shifts and relaxation times.
